(19) United States
(12) Patent Application Publication
(10) Pub. No.: US 2009/0136482 A1
(43) Pub. Date: May 28, 2009

(54) DRUG TARGET PROTEIN AND TARGET GENE, AND SCREENING METHOD

(75) Inventors: Yorimasa Suwa, Tokyo (JP); Tadakazu Yamauchi, Shizuoka (JP); Morikazu Kito, Kanagawa (JP); Yuko Isono, Kanagawa (JP); Tsuruki Tamura, Ibaraki (JP); Motoi Tobita, Tokyo (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: REVERSE PROTEOMICS RESEARCH INSTITUTE CO., LTD., Tokyo (JP)

(21) Appl. No.: 12/065,303

(22) PCT Filed: Sep. 1, 2006
(86) PCT No.: PCT/JP2006/317801

§ 371 Date: Jun. 9, 2008

(30) Foreign Application Priority Data
Sep. 2, 2005 (JP) 2005-255471

(51) Int. Cl. A61K 39/395; G01N 33/00; C12Q 1/68; G01N 33/566; A61K 38/02; A61K 31/7088; A61K 31/7105

(52) U.S. Cl. 424/130.1; 436/86; 436/94; 435/6; 436/501; 514/2; 514/44

(57) ABSTRACT

The present invention provides target proteins and target genes for bioactive substances such as drugs, and means that enable the development of novel bioactive substances using the same. More specifically, the present invention provides target proteins and target genes for bioactive substances; screening methods for substances capable of regulating bioactivities; bioactivity regulators; a bioactive substance derivative production method; a complex comprising a bioactive substance and a target protein, and a method of producing the complex; and kits comprising a bioactive substance or a salt thereof; determination methods for the onset or risk of onset of a specified disease or condition, determination methods for susceptibility to a bioactive substance, and determination kits used for the determination methods, and the like.

### TECHNICAL FIELD

The present invention relates to target proteins and target genes that are useful for the development of bioactive substances, for example, drug discovery; a screening method for a bioactive substance and the substance obtained by the screening method; a bioactivity regulator; a bioactive substance derivative and a method of producing the derivative; and a complex comprising a bioactive substance and a target protein therefor and a method of producing the complex, and the like.

### BACKGROUND ART

Traditionally, the success rate of new drug research and development is quite low, with only one or two of about 100 research projects ending successfully with the launch of a new drug (D. Brown and G. Superti-Furga, Drug Discovery Today, December, 2003). This is mostly because of premature termination of the development due to a problem with the economy, safety or efficacy of the new drug candidate compound (Dimasi, Clin. Pharmacol. Ther., 69, 297-307, 2001).

Pharmaceutical companies are spending 10 to 20% of their sales on R&D activities; it is of paramount importance to efficiently spend R&D budgets for pharmaceutical companies to be highly competitive. Furthermore, because about 80% of R&D expenditures are spent for costly clinical studies in the developmental stage, it is critical to select appropriate candidate compounds in the initial stage prior to progress to the developmental stage.

In recent years, on the other, the genome sequences of a variety of organisms have been elucidated and analyzed at the global level. For the human genome, in particular, a worldwide cooperative research project was implemented, and completion of analysis of all sequences thereof was announced in April 2003. As a result, it is becoming possible to analyze complex biological phenomena in the context of the functions and control of all genes, or networks of gene-gene, protein-protein, cell-cell, and individual-individual interactions. The genome information thus obtained has been significantly revolutionizing a number of industries, including drug development, as well as in academic sectors.

For example, it has been reported that there are about 480 kinds of target proteins for drugs having been in common use to date, and that these target proteins are limited to membrane receptors, enzymes, ion channels, or nuclear receptors and the like (J. Drews, Science, 297, 1960-1964, 2000). Meanwhile, target protein search based on genome information has discovered an extremely large number of target proteins, including novel proteins not covered in the conventional range of target proteins one after another, which are estimated to total about 1,500 kinds (A. L. Hopkins & C. R. Groom, Nature Reviews; Drug Discovery, 1, 727-730, 2002).

However, despite the fact that the research and development expenditures spent by pharmaceutical companies are increasing due to rises in infrastructuring costs for coping with vast amounts of data like genome information and clinical developmental costs, the number of new drugs approved per year is tending to decrease on the contrary (Nature Reviews; Drug Discovery, February, 2003). This shows that the above-described genome information is actually not efficiently utilized.

As a means for overcoming these circumstances, Nagashima et al. invented “Method, System, Apparatus, and Device for Discovering and Preparing Chemical for Medical and Other Uses” and filed a patent application for that invention (National Publication of Translated Version No. 2004-509406).

Disclosed in that patent application are methods, systems, databases, user interfaces, software, media, and services that are useful for the evaluation of compound-protein interactions, and are also useful for the utilization of the information resulting from such an evaluation intended to discover compounds in medical and other areas. Furthermore, it is intended to produce a very large pool of novel target proteins for drug discovery, novel methods for designing novel drugs, and a pool of small substances for therapeutic purposes that are virtually synthesized as having been inconceivable in the past.

Specifically, disclosed in that patent application was a method of identifying a protein or partial protein that is appropriate as a novel drug discovery target, which comprises the following steps:

(i) a step for selecting a plurality of proteins or partial proteins showing desired affinity and specificity for a selected target compound;
(ii) a step for identifying the structure and function of the protein or the partial protein; and
(iii) a step for selecting a single protein or single partial protein having a desired function, and a method of discovering a drug, which comprises the following steps:
(i) a step for investigating the chemical structure of the target compound selected using the above-described method; and
(ii) a step for chemically modifying the structure of the selected target compound to optimize the affinity and specificity of the modified compound for the protein or the partial protein, which is appropriate as a novel drug target.

Furthermore, another feature of the method disclosed in that patent application resides in that the selected target compound is a compound approved for medical use.

Conventional drugs that have been used to date include many drugs for which target proteins are unknown, or for which target proteins are known but not all of whose pharmacological effects and adverse effects can be explained by mechanisms mediated by the proteins.

Typically, aspirin, one of the drugs that have longest been used, may be mentioned. When aspirin was launched in the market for the first time more than 100 years ago, the mechanism for its anti-inflammatory action was unclear. About 70 years later, aspirin was found to have cyclooxygenase (COX) inhibitory action. Still 20 years later, it was demonstrated that COX occurred in two subtypes: COX-1 and COX-2, that the primary pharmacological effect of aspirin was based on COX-2 inhibition, and that COX-1 inhibitory action was the cause of adverse effects such as gastrointestinal disorders. However, not all the target proteins for aspirin have been elucidated. In recent years, aspirin has been shown to exhibit anticancer action and antidementic action in clinical settings, but these pharmacological effects cannot be explained by COX inhibition. On the other, recent years have seen many papers reporting that aspirin acts on transcription factors such as IKKβ and on nuclear receptors such as PPAR-γ, but the association of these and the various pharmacological effects of aspirin remains unclear.

For these reasons, elucidating target proteins for traditionally used drugs can be said to be a very effective approach to discovering novel drug discovery target proteins.

Hirayama, one of the inventors of the above-described published patent, and others generated a database integrating the structural and physical property data on about 1,500 kinds of drugs commercially available in Japan, and found that existing pharmaceutical compounds share structural features (Chem-Bio Informatics Journal, 1, 18-22, 2001). Drugs that have been commonly used to date can be described as excellent in that they have cleared the issues of localization in the body and safety in their developmental processes. Searching novel target proteins with these existing drugs as probes, and selecting novel drug candidate compounds on the basis of their structures is thought to be a highly reasonable and efficient approach.

A second problem arises concerning how to make use of the genome information during the search for novel target proteins. Solely determining the genome sequence is not sufficient to ensure the elucidation of the functions of all genes and the discovery of drug discovery target proteins. It is estimated that in humans, about 30,000 to 40,000 kinds of genes are present; taking into consideration variants from alternative splicing, there are reportedly more than 100,000 kinds of mRNA. It is important, therefore, that out of the vast amount of new genes revealed from the genome sequence, those having useful functions in industrial applications, including drug development, should be efficiently selected and identified.

In many cases, in the genome sequences of eukaryotic organisms, each gene is divided into a plurality of exons by introns; therefore, it is impossible to accurately predict the structure of the protein encoded by the gene solely from the sequence information on the gene. In contrast, for a cDNA prepared from intron-excluded mRNA, information on the amino acid sequence of protein is obtained as information on a single continuous sequence, enabling easy determination of the primary structure thereof.

In particular, analyzing a full-length cDNA enables the identification of the mRNA transcription initiation point on the genome sequence based on the 5′-terminal sequence of the cDNA, and also enables analysis of the stability of mRNA contained in the sequence and of factors involved in expression control in the translation stage. Also, because the ATG codon, which serves as the translation initiation point, is present on the 5′ side, translation into protein in the right frame can be achieved. Therefore, by using an appropriate gene expression system, it is also possible to mass-produce the protein encoded by the cDNA, and to express the protein and analyze the biological activity thereof. Hence, it is considered that by performing an analysis using a protein expressed from full-length cDNA, important information that could not be obtained solely by genome sequence analysis is obtained, and that it is possible to discover novel target proteins that do not lie in the conventional category of drug discovery target proteins.

### DISCLOSURE OF THE INVENTION

The objects of the present invention are to provide target proteins and target genes for the development of bioactive substances (e.g., drug discovery), and various means that enable the development of novel bioactive substances using the same and the like.

The present inventors diligently investigated new drug discovery target proteins that can be useful for the development of new drugs or bioactive substances, by analyzing interactions between human proteins and compounds that have been used as drugs or bioactive substances by surface plasmon resonance, and found novel target proteins and novel target genes that are useful for the development of bioactive substances, for example, drug discovery. The present inventors conducted further investigations based on this finding, conceived that substances that regulate the expression or function of these genes are capable of regulating various bioactivities, and that substances capable of regulating various bioactivities are developed by screening substances that regulate the expression or function of these genes, and by derivatizing these bioactive substances so that the expression or function of the target genes therefor can be regulated, and the like, and completed the present invention.

Accordingly, the present invention is as follows:

[1] A method for screening a substance capable of regulating an action associated with bioactive substance X, which comprises determining whether or not a test substance is capable of regulating the expression or function of target protein Y or a gene that encodes the protein, wherein the combination of bioactive substance X and target protein Y is any of the following (a1) to (a5) (hereinafter referred to as “combinations A” as required):
(a1) a combination of thiabendazole and FLJ10368-, FLJ12389- or FLJ12514-derived protein;
(a2) a combination of reserpine and FLJ10368-derived protein;
(a3) a combination of imipenem and FLJ12435- or FLJ14583-derived protein;
(a4) a combination of cephalexin and FLJ12502-, FLJ14583- or FLJ31146-derived protein;
(a5) a combination of aclarubicin and FLJ12514-derived protein.
[2] The method according to [1] above, which comprises the following steps (a) to (c):
(a) a step for bringing the test substance into contact with target protein Y;
(b) a step for measuring the functional level of the protein in the presence of the test substance, and comparing said functional level with the functional level of the protein in the absence of the test substance;
(c) a step for selecting a test substance that alters the functional level of the protein on the basis of the result of the comparison in (b) above.
[3] The method according to [1] above, which comprises the following steps (a) to (c):
(a) a step for bringing the test substance and cells allowing a measurement of the expression of target protein Y or a gene that encodes the protein into contact with each other;
(b) a step for measuring the expression level of the gene in the cells in contact with the test substance, and comparing said expression level with the expression level of the gene in control cells not in contact with the test substance;
(c) a step for selecting a test substance that regulates the expression level of the gene on the basis of the result of the comparison in (b) above.
[4] The method according to [1] above, which comprises the following steps (a) to (c):
(a) a step for bringing the test substance into contact with target protein Y;
(b) a step for measuring the ability of the test substance to bind to the protein;
(c) a step for selecting a test substance capable of binding to the protein on the basis of the result from (b) above.
[5] The method according to [1] above, which comprises the following steps (a) to (c):
(a) a step for bringing the test substance and a target protein Y-binding substance into contact with target protein Y;
(b) a step for measuring the ability of the target protein Y-binding substance to bind to the protein in the presence of the test substance, and comparing said ability with the ability of the target protein Y-binding substance to bind to the protein in the absence of the test substance;
(c) a step for selecting a test substance that alters the ability of the target protein Y-binding substance to bind to the protein on the basis of the result of the comparison in (b) above.
[6] A method for screening a substance capable of regulating a function associated with target protein Y, which comprises comparing the ability of a test substance to bind to target protein Y or the action associated with the test substance, with the ability of bioactive substance X to bind to target protein Y or the action associated with the bioactive substance, wherein the combination of target protein Y and bioactive substance X is any of the following (b1) to (b7) (hereinafter referred to as “combinations B” as required):
(b1) a combination of FLJ10368-derived protein and thiabendazole, reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ12502-derived protein and cephalexin or a derivative thereof capable of binding to the protein;
(b5) a combination of FLJ12514-derived protein and aclarubicin, thiabendazole or a derivative thereof capable of binding to the protein;
(b6) a combination of FLJ14583-derived protein and cephalexin, imipenem or a derivative thereof capable of binding to the protein;
(b7) a combination of FLJ31146-derived protein and cephalexin or a derivative thereof capable of binding to the protein.
[7] A substance obtained by the method according to any one of [1] to [6] above.
[8] An agent of regulating a bioactivity, which comprises a substance obtained by the method according to any one of [1] to [6] above.
[9] An agent of regulating an action associated with bioactive substance X, which comprises a substance that regulates the expression or function of target protein Y or a gene that encodes the protein, wherein the combination of bioactive substance X and target protein Y is any of combinations A.
[10] The agent according to [9] above, wherein the substance that regulates the expression or function of target protein Y or a gene that encodes the protein is a substance that suppresses the expression or function of the gene.
[11] The agent according to [10] above, wherein the substance that suppresses the expression or function of target protein Y or a gene that encodes the protein is an antisense nucleic acid, ribozyme, decoy nucleic acid, siRNA, antibody or a dominant negative mutant, or an expression vector thereof.
[12] The agent according to [9] above, which comprises target protein Y, or an expression vector comprising a nucleic acid that encodes the protein.
[13] An agent of regulating a function associated with target protein Y, which comprises bioactive substance X, wherein the combination of target protein Y and bioactive substance X is any of combinations B.
[14] A method of producing a derivative of bioactive substance X, which comprises derivatizing bioactive substance X so as to be able to regulate the expression or function of target protein Y or a gene that encodes the protein, wherein the combination of bioactive substance X and target protein Y is any of combinations A.
[15] A method of producing a derivative of a substance capable of regulating a function associated with target protein Y, which comprises derivatizing bioactive substance X so as to be able to regulate the ability of bioactive substance X to bind to target protein Y, wherein the combination of target protein Y and bioactive substance X is any of combinations B.
[16] A bioactive substance derivative obtained by the method according to [14] or [15] above.
[17] An agent of regulating a bioactivity, which comprises a bioactive substance derivative obtained by the method according to [14] or [15] above.
[18] A complex comprising bioactive substance X and target protein Y thereof, wherein the combination of bioactive substance X and target protein Y is any of combinations A or combinations B.
[19] A method of producing the complex according to [18] above, which comprises bringing the bioactive substance and the target protein therefor into contact with each other.
[20] A kit comprising the following (i) and (ii):
(i) bioactive substance X or a salt thereof;
(ii) target protein Y, a nucleic acid that encodes the protein, an expression vector comprising the nucleic acid, cells that enable a measurement of the expression of target protein Y or a gene that encodes the protein, or an expression vector comprising the transcription regulatory region of a gene that encodes target protein Y and a reporter gene functionally linked thereto;

wherein the combination of bioactive substance X and target protein Y is any of combinations A or combinations B.

[21] A method for determining the onset or risk of onset of a disease or condition associated with an action of bioactive substance X, or the onset or risk of onset of a disease or condition associated with a function of target protein Y, which comprises the following steps (a) and (b):
(a) a step for measuring the expression level and/or polymorphism of target protein Y or a gene that encodes the protein in a biological sample collected from an animal;
(b) a step for evaluating the onset or likelihood of onset of the disease or condition on the basis of the measured expression level and/or polymorphism;

wherein the combination of bioactive substance X and target protein Y is any of combinations A or combinations B.

[22] A kit for determining the onset or risk of onset of a disease or condition associated with an action of bioactive substance X, or the onset or risk of onset of a disease or condition associated with a function of target protein Y, which comprises the following (i) and (ii):
(i) a means capable of measuring the expression level and/or polymorphism of target protein Y or a gene that encodes the protein;
(ii) a medium recording the relationship between the disease or condition and the expression level and/or polymorphism;

wherein the combination of bioactive substance X and target protein Y is any of combinations A or combinations B.

[23] A method for determining susceptibility to bioactive substance X in a disease or condition associated with an action of bioactive substance X, or a disease or condition associated with a function of target protein Y, which comprises the following steps (a) and (b):
(a) a step for measuring the expression level and/or polymorphism of target protein Y or a gene that encodes the protein in a biological sample collected from an animal;
(b) a step for predicting the effect of the bioactive substance on the basis of the measured expression level and/or polymorphism;

wherein the combination of bioactive substance X and target protein Y is any of combinations A or combinations B.

[24] A kit for determining susceptibility to bioactive substance X in a disease or condition associated with an action of bioactive substance X, or susceptibility to bioactive substance X in a disease or condition associated with a function of target protein Y, which comprises the following (i) and (ii):
(i) a means capable of measuring the expression level and/or polymorphism of target protein Y or a gene that encodes target protein Y;
(ii) a medium recording the relationship between the effect of bioactive substance X and the expression level and/or polymorphism of the gene;

wherein the combination of bioactive substance X and target protein Y is any of combinations A or combinations B.

### BEST MODE FOR CARRYING OUT THE INVENTION

### 1. Target Proteins and Target Genes for Bioactive Substances

The present invention provides target proteins and target genes for the development of bioactive substances.

A bioactive substance means any substance that has an action on the body. The bioactive substance can be an exogenous substance such as a drug, vitamin, herbal medicine ingredient, or food ingredient, and can be an endogenous substance such as a cytokine, growth factor, or hormone. When a given bioactive substance is intended, it is expressed as bioactive substance X as required.

Bioactive substance X includes the bioactive substances capable of regulating the expression or function of target protein Y or a gene that encodes the protein, described below, for example, bioactive substances capable of binding to target protein Y. Specifically, bioactive substance X can be thiabendazole, reserpine, imipenem, cephalexin or aclarubicin, or a derivative thereof capable of binding to target protein Y (described later), or a salt thereof.

Bioactive substances can also be roughly divided, from the viewpoint of the type of activity that can be regulated thereby, into two groups: substances capable of regulating an action associated with bioactive substance X, and substances capable of regulating a function associated with target protein Y.

The target proteins and target genes for the development of bioactive substances can be preferable target proteins and target genes for drug discovery. When a given target protein and a given target gene are intended, they are expressed as target protein Y and target gene Y, respectively, as required. The term protein has the same definition as a translation product, and the term target gene Y has the same definition as a gene that encodes target protein Y; these terms are interchangeably used.

For example, target protein Y can be a target protein for the above-described bioactive substance X. Specifically, target protein Y can be FLJ10368-, FLJ12389-, FLJ12435-, FLJ12502-, FLJ12514-, FLJ14583- or FLJ31146-derived protein.

FLJXXXXX-derived protein can be a human protein having a given FLJ number, or a GenBank accession number, H-Inv cDNA ID or H-Inv locus ID corresponding thereto. Such a protein can be a protein consisting of an amino acid sequence registered as FLJXXXXX, or a GenBank accession number, H-Inv cDNA ID or H-Inv locus ID corresponding thereto (see, for example, Table 1), or a protein comprising (having?) the amino acid sequence (e.g., full-length protein). As mentioned herein, the target proteins of the present invention are not limited to the aforementioned human proteins, but include orthologues of different animal species (e.g., mouse, rat, dog, monkey etc.). FLJ numbers can be retrieved from, for example, FLJ-DB homepage. Referring to human proteins for reference, information on various aspects and some examples of binding bioactive substances discovered by the present inventors are shown in Table 1, respectively.

TABLE 1

| | | | | Examples |
|---|---|---|---|---|
| | GenBank | | | of bound |
| | Accession | H-InV cDNA | H-Inv locus | bioactive |
| FLJ No. | No. | ID | ID | substances |
| FLJ10368 | AK001230 | HIT000003704 | HIX0007044 | thiabendazole |
| | | | | reserpine |
| FLJ12389 | AK022451 | HIT000005725 | HIX0022598 | thiabendasole |
| FLJ12435 | AK022497 | HIT000005771 | HIX0001107 | imipenem |
| FLJ12502 | AK022564 | HIT000005838 | HIX0011889 | cephalexin |
| FLJ12514 | AK022576 | HIT000005850 | HIX0018971 | aclarubicin |
| | | | | thiabendazole |
| FLJ14583 | AK027489 | HIT000010761 | HIX0016824 | cephalexin |
| | | | | imipenem |
| FLJ31146 | AK055708 | HIT000012322 | HIX0006596 | cephalexin |

The present invention also provides variant proteins, having bioactivity, of FLJXXXXX-derived proteins (hereinafter abbreviated where necessary as variant proteins; FLJXXXXX-derived proteins or variant proteins thereof having bioactivity are also abbreviated where necessary simply as FLFXXXXX-derived proteins). The variant protein is not particularly limited as long as it has bioactivity that can be regulated by interaction with a bioactive substance; and, for example, artificial mutants or natural mutants (e.g., splicing variants, or proteins having polymorphism such as SNP, haplotype or the like) can be mentioned.

Specifically, such a variant protein can be a protein consisting of an amino acid sequence resulting from the substitution, deletion, addition or insertion of one or more amino acids in the amino acid sequence encoded by FLJ10368-, FLJ12389-, FLJ12435-, FLJ12502-, FLJ12514-, FLJ14583- or FLJ31146-derived protein, and that has bioactivity that can be regulated by an interaction with a bioactive substance. The number of amino acids substituted, deleted, added or inserted can be any one that allows the retention of the function, for example, about 1 to 50, preferably about 1 to 30, more preferably about 1 to 20, further more preferably about 1 to 10, most preferably 1 to 5 or 1 or 2. The site for substitution, deletion, addition or insertion of an amino acid can be any site that allows the retention of the function, for example, a site other than functionally important domains.

Furthermore, a variant protein can be a protein which consists of, for example, an amino acid sequence having a homology of about 50% or more, preferably about 70% or more, more preferably about 80% or more, further more preferably about 90% or more, most preferably about 95% or more (but excluding 100% homology), to the amino acid sequence encoded by FLJ10368-, FLJ12389-, FLJ12435-, FLJ12502-, FLJ12514-, FLJ14583- or FLJ31146-derived protein, and which has bioactivity that can be regulated by an interaction with a bioactive substance. Here, the numerical values of the above-described homology are calculated by, for example, executing the commands for the maximum matching method using the DNASIS sequence analytical software (Hitachi Software Engineering). The parameters for the calculation should be used in default settings (initial settings).

When a target protein of the present invention is used, the protein may be a labeled supply or a non-labeled supply, or a mixture of a labeled supply and a non-labeled supply mixed in a specified ratio. Examples of the labeling substance include fluorescent substances such as FITC and FAM, luminescent substances such as luminol, luciferin and lucigenin, radioisotopes such as ³H, ¹⁴C, ³²P, ³⁵S, and ¹²³I, affinity substances such as biotin and streptavidin, and the like.

The target genes of the present invention may be any ones that encode the target proteins of the present invention. For example, the target genes of the present invention can be those corresponding to proteins comprising the above-described amino acid sequences. For example, proteins comprising the above-described amino acid sequences can be those corresponding to cDNA clones having nucleotide sequences corresponding to the FLJ nucleotide sequence accession numbers shown in Table 1. In the H-Invitational Database (H-InvDB), for example, cDNA clones that share a gene region on the human genome are classified as a cluster; the cDNA clones corresponding to the proteins of the present invention are given respective H-Inv cDNA IDs shown in Table 1, and the gene loci thereof are given respective H-Inv locus IDs. Hence, the target genes of the present invention can be cDNAs of the FLJ nucleotide sequence accession numbers shown in Table 1, a cDNA cluster of H-Inv cDNA IDs in H-InvDB, or genes given H-Inv locus IDs or variant genes thereof.

The target genes of the present invention can be a gene that consists of a nucleotide sequence that hybridizes to a sequence complementary to the nucleotide sequence corresponding to one of the FLJ nucleotide sequence accession numbers shown in Table 1 under stringent conditions, and that corresponds to a protein that interacts with a bioactive substance. Here, “hybridize under stringent conditions” means that a positive hybridization signal remains observable even under conditions of, for example, heating in a solution of 6×SSC, 0.5% SDS and 50% formamide at 42° C., followed by washing in a solution of 0.1×SSC and 0.5% SDS at 68° C.

The target proteins and target genes of the present invention can be used for the development of drugs for diseases or conditions associated with bioactive substance X, or diseases or conditions associated with target gene Y (or target protein Y), or for the development of investigational reagents for the diseases or conditions, and the like. Diseases or conditions associated with bioactive substance X and diseases or conditions associated with target gene Y are described in detail below.

(Diseases or Conditions Associated with Bioactive Substance X)

“A disease or condition associated with bioactive substance X” means a disease for which bioactive substance X is used or a disease corresponding to an adverse effect of bioactive substance X, or a condition for which use of bioactive substance X is desired (e.g., a deficiency of bioactive substance X) or an unwanted condition caused by bioactive substance X (e.g., an unwanted condition caused by excess intake of bioactive substance X). A disease or condition associated with bioactive substance X can be ameliorated or exacerbated by bioactive substance X.

“An action associated with bioactive substance X” means an action of the same kind as, or opposite kind to, a kind of action actually exhibited by bioactive substance X (including pharmacological actions and adverse effects). Hence, an action associated with bioactive substance X is an action capable of ameliorating or exacerbating “a disease or condition associated with bioactive substance X”. For example, “an action associated with bioactive substance X” is a hypotensive action, a hypertensive action and the like when bioactive substance X is reserpine.

“A disease or condition associated with bioactive substance X” and “an action associated with bioactive substance X” vary depending on the kind of bioactive substance X. Described below are “diseases or conditions associated with bioactive substance X” with reference to substances that represent bioactive substance X. Because “an action associated with bioactive substance X” is any action capable of ameliorating or exacerbating “a disease or condition associated with bioactive substance X”, the following description of “diseases or conditions associated with bioactive substance X” will surely lead to the clarification of “actions associated with bioactive substance X”.

### Thiabendazole

A disease associated with thiabendazole means a disease for which thiabendazole is used or a disease corresponding to an adverse effect of thiabendazole. Thiabendazole is known as an anthelmintic and the like. Examples of the disease for which thiabendazole is applied include parasitism due to Strongyloides stercoralis, and the like. On the other hand, examples of the adverse effect of thiabendazole include confusion, hallucination, abnormal excitation, spasm, severe diarrhea, shock, skin blister, skin avulsion, intense itching and eruption, slight chill, chill, fever onset, muscular pain, arthritic pain, abnormal malaise, weakness, blurry vision, yellow vision, brown urine, hematuria, lumbar backache, yellow skin and yellow eye, whitish stool, dizziness, staggering sensation, tinnitus, headache, dry eye and dry mouth, loss of appetite and the like. An action associated with thiabendazole can be closely relevant to a target protein (target gene) therefor, for example, FLJ10368-, FLJ12389- or FLJ12514-derived protein.

### Reserpine

A disease associated with reserpine means a disease for which reserpine is used or a disease corresponding to an adverse effect of reserpine. Reserpine is known as a therapeutic agent for hypertension, therapeutic agent for psychoneurotic disease and the like. Examples of the disease for which reserpine is used include hypertension (essential), hypertension (renal and the like), malignant hypertension (in combination with other hypotensive agents), schizophrenia for which phenothiazine-series drugs are difficult to use, and the like. On the other hand, examples of the adverse effect of reserpine include depressive states, nightmares, drowsiness, decrease in sexuality, nervous irritability, vertigo, headache, systemic tremor, extrapyramidal symptoms, eruption, bradycardia, edema, gastric ulcer, dry mouth, diarrhea, loss of appetite, nausea, vomiting, soft feces, nasal obstruction, malaise, dyspnoea, body weight increase and the like. As targets of reserpine, synaptic vesicle amine transporter and actin are known. An action associated with reserpine can be closely relevant to a target protein (target gene) therefor, for example, FLJ10368-derived protein. imipenem

A disease associated with imipenem means a disease for which imipenem is used or a disease corresponding to an adverse effect of imipenem. Imipenem is known as a carbapenem series antibiotic preparation and the like. Examples of bacterial species for which imipenem is used include imipenem-sensitive Staphylococcus, Streptococcus, Pneumococcus, Enterococcus, Escherichia coli, Citrobacter, Klebsiella, Enterobactor, Serratia, Proteus, Morganella morganii, Providencia, Haemophilus influenzae, Pseudomonas, Pseudomonas aeruginosa, Burkholderia cepacia, Acinetobacter, Peptostreptococcus, Bacteroides, Prevotella and the like. Examples of the disease for which imipenem is used include sepsis, infectious endocarditis, secondary infection after trauma, burn, surgical-wound and the like, osteomyelitis, arthritis, acute bronchitis, pneumonia, lung abscess, thoracic empyema, secondary infection of chronic respiratory pathology, cystitis, pyelonephritis, prostatitis (acute, chronic), peritonitis, cholecystitis, cholangitis, purohepatitis, bartholinitis, intrauterine infection, uterine adnexitis, parametritis, keratitis (including corneal ulcer), intraocular inflammation (including panophthalmitis) and the like. On the other hand, examples of the adverse effect of imipenem include convulsion, asphyxia, disturbed consciousness, unconsciousness, respiratory depression, confusion, disquietness, shock, anaphylactoid symptom, mucocutaneous ocular syndrome (Stevens-Johnson syndrome), toxic epidermal necrosis, fulminant hepatitis, hepatitis, hepatic failure, jaundice, attack and induction of asthma, fever onset, coughing, dyspnea, breast X-ray abnormality, interstitial pneumonia accompanied by eosinophilia, PIE syndrome, pancytopenia, bone marrow suppression, agranulocytosis, hemolytic anemia, acute renal failure, diabetes insipidus, pseudomembranous colitis, thrombophlebitis, eruption, itching, fever onset, urticaria, flush, erythema, granulocytopenia, eosinophilia, basophilia, lymphocytosis, thrombocytopenia and thrombocytosis, erythropenia, hypochromic erythrocyte, decrease in hematocrit, elevation in AST (GOT), elevation in ALT (GPT), elevation in LDH, elevation in Al—P, elevation in γ-GTP, elevation in urinary urobilinogen, oliguria, elevation in BUN, elevation in serum creatinine, pollakiuria, elevation in serum amylase, discoloration of tongue, abdominal pain, diarrhea, nausea, vomiting, loss of appetite, hallucination, delirium, numbness, tremor, stomatitis, candidiasis, vitamin K deficiency symptom (hypoprothrombinemia, breeding tendency etc.), vitamin B family deficiency symptom (glossitis, stomatitis, loss of appetite, neuritis etc.), headache, malaise, edema, chest pain, taste abnormality, pain and induration at injection site, lowering of serum sodium, elevation and lowering of serum potassium and the like. As targets of imipenem, penicillin-binding protein (PBPs) 2 and sodium-dependent dopamine transporter are known. An action associated with imipenem can be closely relevant to a target protein (target gene) therefor, for example, FLJ12435- or FLJ14583-derived protein. cephalexin

A disease associated with cephalexin means a disease for which cephalexin is used or a disease corresponding to an adverse effect of cephalexin. Cephalexin is known as a long-acting cephem-series oral antibiotic preparation and the like. Examples of bacterial species to which cephalexin is used include cephalexin-sensitive Staphylococcus, Streptococcus, Pneumococcus, Escherichia coli, Klebsiella, Proteus mirabilis and the like. Examples of the disease for which cephalexin is used include superficial skin infections, deep skin infections, lymphangitis and lymphadenitis, chronic pyoderma, secondary infection after trauma, burn, surgical-wound and the like, mastitis, laryngopharyngitis, tonsillitis (including peritonsillitis), acute bronchitis, pneumonia, secondary infection of chronic respiratory pathology, cystitis, pyelonephritis, prostatitis (acute, chronic), bartholinitis, dacryocystitis, hordeolum, otitis externa, otitis media, sinusitis, inflammation around tooth tissue, pericoronitis, jaw inflammation, second infection of extraction wound and oral surgery wound, and the like. On the other hand, examples of the adverse effect of cephalexin include shock, anaphylactoid symptoms, acute renal failure, hemolytic anemia, pseudomembranous colitis, mucocutaneous ocular syndrome (Stevens-Johnson syndrome), toxic epidermal necrosis (Lyell syndrome), interstitial pneumonia, PIE syndrome, eruption, urticaria, erythema, itching, fever onset, lymphadenopathy, arthritic pain, granulocytopenia, eosinophilia, thrombocytopenia, jaundice, elevation in AST (GOT), elevation in ALT (GPT), elevation in Al—P, nausea, vomiting, diarrhea, loose stool, abdominal pain, loss of appetite, gastric distress, stomatitis, candidiasis, vitamin K deficiency symptom (hypoprothrombinemia, breeding tendency etc.), vitamin B family deficiency symptom (glossitis, stomatitis, loss of appetite, neuritis etc.), headache, dizziness, general malaise and the like. As a target of cephalexin, organic anion transporter 1 is known. An action associated with cephalexin can be closely relevant to a target protein (target gene) therefor, for example, FLJ12502-, FLJ14583- or FLJ31146-derived protein. aclarubicin

A disease associated with aclarubicin means a disease for which aclarubicin is used or a disease corresponding to an adverse effect of aclarubicin. Aclarubicin is known as an antitumor antibiotic and the like. Examples of the disease for which aclarubicin is used include remission and improvement of subjective and objective symptoms of gastric cancer, lung cancer, breast cancer, ovarian cancer, malignant lymphoma and acute leukemia, and the like. On the other hand, examples of the adverse effect of aclarubicin include myocardial cell disorder, cardiac failure, bone marrow suppression, pancytopenia, anemia, leucocytopenia, thrombocytopenia, bleeding, electrocardiogram abnormality, tachycardia, arrhythmia, loss of appetite, nausea, vomiting, stomatitis, diarrhea, gastrointestinal bleeding, abdominal pain, elevation in AST (GOT), elevation in ALT (GPT), elevation in Al—P, proteinuria, hematuria, cystitis, urodynia, frequency of urination, feeling of residual urine, eruption, hair loss, pigmentation, general malaise, headache, heaviness of the head, pancreatitis, fever onset, phlebitis, face flush and the like. As a target of aclarubicin, DNA topoisomerase II is known. An action associated with aclarubicin can be closely relevant to a target protein (target gene) therefor, for example, FLJ12514-derived protein.

(Diseases or Conditions Associated with Target Gene Y)

“A disease or condition associated with target gene Y” refers to a disease or condition that can be caused as a result of a functional change (e.g., functional changes due to mutations (e.g., polymorphism)), or a change in the expression level, in target gene Y, or in a gene located downstream of target gene Y in the signal transduction system mediated by target gene Y (downstream gene). A functional change in target gene Y or a gene downstream thereof can be caused by, for example, a mutation (e.g., polymorphism) in the gene. Examples of the mutation include a mutation in the coding region, which promotes or suppresses a function of the gene, a mutation in the non-coding region, which promotes or suppresses the expression thereof, and the like. The change in the expression level includes increases or reductions in the expression level. A disease or condition associated with target gene Y can be ameliorated or exacerbated by target protein Y.

“A function associated with target protein Y (target gene Y)” means a function of the same kind as, or opposite kind to, the kind of a function that is actually exhibited by target protein Y. In other words, a function associated with target protein Y is a function capable of ameliorating or exacerbating “a disease or condition associated with target protein Y”. Hence, “a function associated with target protein Y” is a function for promoting or suppressing an immune reaction, and the like, if target protein Y is a factor that promotes an immune reaction.

Since target gene Y is considered to mediate a wide variety of physiological functions in the body; as diseases or conditions associated with target protein Y, a very wide variety of diseases or conditions are supposed. Examples of the diseases or condition associated with target protein Y include diseases or conditions associated with the functions shown in Table 2.

Other examples of the disease or condition associated with target protein Y are diseases or conditions postulated from the annotation of target protein Y and target gene Y. Those skilled in the art can postulate such diseases or conditions by identifying homologous proteins or genes by homology search, and subsequently extensively examining the functions of the proteins or genes or the diseases or conditions mediated thereby by a commonly known method. Various methods are available for annotation analysis. Described below are the results of annotation of target genes for bioactive substances in the present application, by various methods using the sequences of human proteins or genes representative of target proteins or genes for bioactive substances as query sequences.

### Amino Acid Analysis 1

### Homology Analysis by BLASTP

The calculation program used was blastall 2.2.6. The target databases used were swiss-prot: 146720 (2004.03.29), (Refseq)hs: 21170 (2004.05.06), (Refseq)mouse: 17089 (2004.05.06), and (Refseq)rat: 4893 (2004.05.06). The cutoff value was established at 1.00E-05. The following data were processed by filtering:

### For Swiss-Prot:

Having a definition beginning with “ALU SUBFAMILY”

Having a definition beginning with “Alu subfamily”

Having a definition beginning with “!!!! ALU SUBFAMILY”

Having a definition beginning with “B-CELL GROWTH FACTOR PRECURSOR”

Having a definition including “NRK2”

Having a definition beginning with “PROLINE-RICH”

Having a definition beginning with “GLYCINE-RICH”

Having a definition beginning with “EXTENSIN PRECURSOR”

Having a definition beginning with “COLLAGEN”

Having a definition beginning with “100KD”

Having a definition beginning with “RETROVIRUS-RELATED POL POLYPROTEIN”

Having a definition beginning with “CUTICLE COLLAGEN”

Having a definition beginning with “HYPOTHETICAL”

Having a definition beginning with “Hypothetical”

Having a definition beginning with “SALIVARY PROLINE-RICH PROTEIN”

Having a definition beginning with “IMMEDIATE-EARLY PROTEIN”

Having the accession number “P49646”

### For Ref-Seq:

Having a definition beginning with “hypothetical protein FLJ”

Having a definition beginning with “KIAA”

Having a definition beginning with “hypothetical protein DKFZ”

Having a definition beginning with “DKFZ”

Having a definition beginning with “RIKEN cDNA”

Having a definition beginning with “hypothetical protein MGC”

Having a definition as “hypothetical protein”

Having a definition beginning with “hypothetical protein PP”

Having a definition as “neuronal thread protein”

Having a definition beginning with “clone FLB”

Having a definition beginning with “hypothetical protein PRO”

Having a definition as “PRO0483 protein”

Having a definition including “MNC”

Having a definition including “MOST-1”

Having a definition beginning with “similar to”

Having a definition including “TPR gene on Y”

Having a definition beginning with “HSPC”

Having a definition beginning with “CGI-”

ReFSeq sequence composed of self only (information referenced from LL_tmpl)

The annotation information obtained by this analysis is shown in Tables 2-1 to 2-2.

TABLE 2-1

| FLJ No. | Accession No. and definition | Key words |
|---|---|---|
| FLJ10368 | Q9NUX5 Protection of telomeres 1 (hPot1) | Alternative |
| | (POT1-like telomere end-binding protein) | splicing; |
| | Q95K48 Protection of telomeres 1 (POT1-like | Chromosomal |
| | telomere end-binding protein) | Protein; DNA-binding; |
| | Q91WC1 Protection of telomeres 1 (mPot1) | Nuclear |
| | (POT1-like telomere end-binding protein) | protein; Telomere. |
| | Q9NUX5 Protection of telomeres 1 (hPot1) | |
| | (POT1-like telomere end-binding protein) | |
| | NP_056265.1 Protection of telomeres 1 | |
| | [Human] | |
| | NP_598692.1 POT1-like telomere end-binding | |
| | protein [Mouse] | |
| | NP_056265.1 Protection of telomeres 1 | |
| | [Human] | |
| FLJ12389 | Q9Z3R3 Acetoacetyl-coenzyme A synthetase | Acetylation; |
| | (EC6.2.1.16) (Acetoacetate-CoA ligase 1) | Complete |
| | (Acyl-activating enzyme 1) | proteome; |
| | O93730 Acetyl-coenzyme A synthetase (EC6.2.1.1) | Ligase. |
| | (Acetate-CoA ligase) (Acyl- | |
| | activating enzyme) | |
| | Q9QXG4 Acetyl-coenzyme A synthetase, | |
| | cytoplasmic (EC6.2.1.1) (Acetate-CoA | |
| | ligase) (Acyl-activating enzyme) (Acetyl-CoA | |
| | synthetase) (ACS) (AceCS) | |
| | Q9Z3R3 Acetoacetyl-coenzyme A synthetase | |
| | (EC6.2.1.16) (Acetoacetate-CoA ligase 1) | |
| | (Acyl-activating enzyme 1) | |
| | NP_076417.2 Acetoacetyl-CoA synthetase | |
| | [Human] | |
| | NP_084486.1 Acetoacetyl-CoA synthetase | |
| | [Mouse] | |
| | NP_075592.1 Acetoacetyl-CoA synthetase | |
| | [Rat] | |
| | NP_076417.2 Acetoacetyl-CoA synthetase | |
| | [Human] | |
| FLJ12435 | Q9JI55 Plectin 1 (PLTN) (PCN) (300-kDa | Actin-binding; |
| | intermediate filament-associated protein) | Coiled coil; |
| | (IFAP300) (Fragment) | Cytoskeleton; |
| | Q9JI55 Plectin 1 (PLTN) (PCN) (300-kDa | Phosphorylation; |
| | intermediate filament-associated protein) | Repeat; |
| | (IFAP300) (Fragment) | Structural |
| | NP_065385.2 Pre-B-cell leukemia | protein. |
| | transcription factor interacting protein 1 | |
| | [Human] | |
| | NP_666243.1 Pre-B-cell leukemia | |
| | transcription factor interacting protein 1 | |
| | [Mouse] | |
| | NP_065790.1 Cell cycle progression 1 | |
| | [Human] | |
| | NP_065385.2 Pre-B-cell leukemia | |
| | transcription factor interacting protein 1 | |
| | [Human] | |
| FLJ12502 | Q9H8Y9 Protein C14orf159, mitochondrial | Alternative |
| | precursor (UNQ2439/PRO5000) | splicing; |
| | Q8BH86 Protein C14orf159 homolog, | Mitochondrion; |
| | mitochondrial precursor | Transit peptide. |
| | Q9H8Y9 Protein C14orf159, mitochondrial | |
| | precursor (UNQ2439/PRO5000) | |
| | NP_079228.3 Chromosome 14 open reading | |
| | frame 159 [Human] | |
| | NP_079228.3 Chromosome 14 open reading | |
| | frame 159 [Human] | |
| FLJ12514 | NP_056257.1 CCR4-NOT transcription complex, | |
| | subunit 10 [Human] | |
| | NP_705813.2 CCR4-NOT transcription complex, | |
| | subunit 10 [Mouse] | |
| | NP_056257.1 CCR4-NOT transcription complex, | |
| | subunit 10 [Human] | |

TABLE 2-2

| | | |
|---|---|---|
| FLJ14583 | Q12816 Trophinin (MAGE-D3 antigen) | Antigen; Cell |
| | Q9UNF1 Melanoma-associated antigen D2 (MAGE- | adhesion; |
| | D2 antigen) (MAGE-D) (Breast cancer | Repeat; |
| | associated gene 1 protein) (BCG-1) (11B6) | Alternative |
| | (Hepatocellular carcinoma-associated protein | splicing; |
| | JCL-1) | Multigene |
| | Q96JG8 Melanoma-associated antigen D4 (MAGE- | family; Polymorphism. |
| | D4 antigen) (MAGE-E1 antigen) | |
| | Q12816 Trophinin (MAGE-D3 antigen) | |
| | NP_808224.1 Trophinin isoform 2 [Human] | |
| | NP_057241.2 Trophinin isoform 2 [Human] | |
| | NP_808223.1 Trophinin isoform 1 [Human] | |
| | NP_808224.1 Trophinin isoform 2 [Human] | |
| FLJ31146 | Q9JJK5 Homocysteine-responsive endoplasmic | Endoplasmic |
| | reticulum-resident ubiquitin-like domain | reticulum; |
| | member 1 protein | Unfolded |
| | Q15011 Homocysteine-responsive endoplasmic | protein |
| | reticulum-resident ubiquitin-like domain | response; |
| | member 1 protein (Methyl | Alternative |
| | methanesulfonate(MMF)-inducible fragment | splicing; |
| | protein 1) | Transmembrane. |
| | Q9JJK5 Homocysteine-responsive endoplasmic | |
| | reticulum-resident ubiquitin-like domain | |
| | member 1 protein | |
| | NP_071726.1 Homocysteine-inducible, | |
| | endoplasmic reticulum stress-inducible, | |
| | ubiquitin-like domain member 1 [Mouse] | |
| | NP_445975.1 Homocysteine-inducible, | |
| | endoplasmic reticulum stress-inducible, | |
| | ubiquitin-like domain member 1 [Rat] | |
| | NP_055500.1 Homocysteine-inducible, | |
| | endoplasmic reticulum stress-inducible, | |
| | ubiquitin-like domain member 1 isoform 1 | |
| | [Human] | |
| | NP_071768.2 Hypothetical protein FLJ22313 | |
| | [Human] | |

### Amino Acid Analysis 2

### Motif Analysis by Pfam

The calculation program used was hmmpfam (v2.3.2). The target databases used were Pfam DB entry: 7426 families (Pfam13.0, Pfam_{—}1s). (April 2004). The cutoff value was established at 1E-10. The annotation information obtained by this analysis is shown by Table 3.

TABLE 3

| | PfamID and Pfam name | | Pfam description |
|---|---|---|---|
| FLJ10368 | PF02765.7 | Telo_bind | Telomere-binding protein α |
| | | | subunit, central domain |
| FLJ12389 | PF00501.14 | AMP-binding | AMP-binding enzyme |
| FLJ12435 | | | |
| FLJ12502 | PF07286.2 | DUF1445 | Protein of unknown |
| | | | function (DUF1445) |
| FLJ12514 | | | |
| FLJ14583 | PF01454.7 | MAGE | MAGE family |
| FLJ31146 | | | |

### Amino Acid Analysis 3

### Prediction of Secretory Signal Sequences by Signal IP

The calculation program used was SignalP ver 3.0 (May 18, 2004).

### Amino Acid Analysis 4

### Functional Categorization by GeneOntology

Performed per the procedures described below.

1) Extract results having E-values that meet the following conditions from among the results of homology analysis using BLASTP (RefSeq and SwissProt with filter) that produced three higher BLAST results (six in total).
Condition 1: Use all results having E-values of not more than 1E-50.
Condition 2: Do not use results having E-values of not less than 1E-10.
Condition 3: Use results having E-values exceeding 1E-50, provided that the difference in E-value from Top Hit is within 1E+20.
Condition 4: If the E-value of Top Hit is 0, use results having E-values of not more than 1E-50.
2) Search GO by the keywords of SwissProt using spkw2go.
3) Search xref.goa by accession numbers of SwissProt to acquire Refseq IDs, further acquire LOCUS IDs by the Refseq IDs using LL_tmpl, and acquire GO terms by the LOCUS IDs using loc2go.
4) Acquire LOCUS IDs by accession numbers of Refseq using LL_tmpl, and acquire GO terms by the LOCUS IDs using loc2go.
5) Acquire information on higher categories for each GO term acquired, with reference to the Molecular Function text file, Biological Process text file, and Cellular Component text file.
6) Remove overlapping information from the GO term information acquired in 1)-5) above, and make an output.

The annotation information obtained by this analysis is shown in Table 4.

TABLE 4

| | GO | |
|---|---|---|
| FLJ No. | category | GO No. (term) |
| LJ10368 | MF | GO: 0003676¥MF\|nucleic acid binding |
| | | GO: 0003677¥MF\|DNA binding |
| | | GO: 0042162¥MF\|telomeric DNA binding |
| | BP | GO: 0006260¥BP\|DNA replication |
| | | GO: 0007004¥BP\|telomerase-dependent |
| | | telomere maintenance |
| | | GO: 0016233¥BP\|telomere capping |
| | CC | GO: 0000781¥CC\|chromosome, telomeric |
| | | region |
| FLJ12389 | MF | GO: 0003824¥MF\|catalytic activity |
| | | GO: 0016405¥MF\|CoA-ligase activity |
| | | GO: 0016874¥MF\|ligase activity |
| | | GO: 0030729¥MF\|acetoacetate-CoA ligase |
| | | activity |
| | BP | GO: 0006629¥BP\|lipid metabolism |
| | | GO: 0006631¥BP\|fatty acid metabolism |
| | | GO: 0008152¥BP\|metabolism |
| | CC | |
| FLJ12435 | MF | GO: 0003714¥MF\|transcription corepressor |
| | | activity |
| | BP | GO: 0016481¥BP\|negative regulation of |
| | | transcription |
| | | GO: 0030154¥BP\|cell differentiation |
| | CC | GO: 0005634¥CC\|nucleus |
| | | GO: 0005829¥CC\|cytosol |
| FLJ12502 | MF | GO: 0003723¥MF\|RNA binding |
| | | GO: 0005525¥MF\|GTP binding |
| | BP | GO: 0006614¥BP\|SRP-dependent |
| | | cotranslational |
| | | protein-membrane targeting |
| | CC | GO: 0005739¥CC\|mitochondrion |
| | | GO: 0005786¥CC\|signal recognition |
| | | particle (sense Eukaryota) |
| FLJ12514 | MF | GO: 0005488¥MF\|binding |
| | | GO: 0005554¥MF\|molecular_function unknown |
| | BP | GO: 0000004¥BP\|biological_process unknown |
| | CC | GO: 0008372¥CC\|cellular_component unknown |
| FLJ14583 | MF | GO: 0005515¥MF\|protein binding |
| | BP | GO: 0007155¥BP\|cell adhesion |
| | | GO: 0007156¥BP\|homophilic cell adhesion |
| | | GO: 0007566¥BP\|embryo implantation |
| | CC | GO: 0005886¥CC\|plasma membrane |
| | | GO: 0005887¥CC\|integral to plasma membrane |
| FLJ31146 | MF | GO: 0005554¥MF\|molecular_function unknown |
| | BP | GO: 0006464¥BP\|protein modification |
| | | GO: 0006950¥BP\|response to stress |
| | | GO: 0006986¥BP\|response to unfolded protein |
| | CC | GO: 0005783¥CC\|endoplasmic reticulum |
| | | GO: 0005789¥CC\|endoplasmic reticulum |
| | | membrane |
| | | GO: 0016021¥CC\|integral to membrane |

### Nucleic Acid Analysis 1

### Homology Analysis 1 by BLASTX

The calculation program used was blastall 2.2.6. The target database used was nr:1552011 (2004.07.16). The cutoff value was established at 1.00E-05. The following data were processed by filtering:

Having a definition beginning with “ALU SUBFAMILY”

Having a definition including “Alu subfamily”

Having a definition beginning with “!!!! ALU SUBFAMILY”

Beginning with “Drosophila melanogaster genomic scaffold”

Beginning with “Human DNA sequence from”

Including “genomic DNA”

Including “BAC clone”

Including “PAC clone”

Including “cosmid”

Including “complete genome”

Ending with “complete sequence”

Including “genomic sequence”

Including “exon”

A “HIT LENGTH (sequence length of the hit sequence) of not less than 50000 obtained by this analysis

The annotation information obtained by this analysis is shown in Tables 5-1 to 5-2.

TABLE 5-1

| FLJ No. | nr accession No. and definition |
|---|---|
| FLJ10368 | dbj\|BAA91568.1\|unnamed protein product |
| | [Human]¥ ref\|NP_056265.1\|protection of telomeres 1 |
| | [Human] |
| | gb\|AAW22613.1\|protection of telomeres protein 1 variant 1 |
| | [Human]¥ dbj\|BAB14110.1\|unnamed protein product |
| | [Human]¥dbj\|BAA91988.1\|unnamed protein product |
| | [Human]¥ gb\|AAH02923.1\|Protection of telomeres 1 |
| | [Human]¥ sp\|Q9NUX5\|POTE1_HUMAN |
| | Protection of telomeres 1 |
| | (hPot1) (POT1-like telomere end-binding protein) |
| | emb\|CAH92225.1\|hypothetical protein [Orangutan] |
| | dbj\|BAB62219.1\|hypothetical protein |
| | [Cynomolgus]¥ sp\|Q95K48\|POTE1_MACFA |
| | Protection of telomeres |
| | 1 (POT1-like telomere end-binding protein) (QtrA-10940) |
| | ref\|XP_519345.1\|PREDICTED: POT1 |
| | protection of telomeres 1 |
| | homolog [Chimpanzee] |
| | dbj\|BAA91568.1\|unnamed protein product |
| | [Human]¥ ref\|NP_056265.1\|protection |
| | of telomeres 1 [Human] |
| FLJ12389 | dbj\|BAB14040.1\|unnamed protein product [Human] |
| | gb\|AAH51862.1\|Acetoacetyl-CoA synthetase |
| | [Human]¥ ref\|NP_076417.2\|acetoacetyl-CoA synthetase |
| | [Human]¥ dbj\|BAD22560.1\|acetoacetyl-CoA |
| | synthetase [Human] |
| | gb\|AAH40490.2\|Acetoacetyl-CoA synthetase [Human] |
| | dbj\|BAB01571.1\|unnamed protein product [Cynomolgus] |
| | dbj\|BAB41151.1\|hypothetical protein [Cynomolgus] |
| | dbj\|BAB14040.1\|unnamed protein product [Human] |
| FLJ12435 | gb\|AAG02026.1\|hematopoietic PBX-interacting protein |
| | [Human] |
| | emb\|CAI13239.1\|pre-B-cell leukemia transcription factor |
| | interacting protein 1 [Human]¥ ref\|NP_065385.2\|pre-B-cell |
| | leukemia transcription factor interacting protein 1 |
| | [Human]¥ gb\|AAH16852.1\|Pre-B-cell leukemia |
| | transcription |
| | factor interacting protein 1 [Human] |
| | dbj\|BAB14059.1\|unnamed protein product [Human] |
| | emb\|CAI13238.1\|pre-B-cell leukemia transcription factor |
| | interacting protein 1 [Human] |
| | dbj\|BAB14471.1\|unnamed protein product [Human] |
| | gb\|AAG02026.1\|hematopoietic PBX-interacting protein |
| | [Human] |
| FLJ12502 | dbj\|BAB14102.1\|unnamed protein product |
| | [Human]¥ sp\|Q7Z3D6\|CN159_HUMAN |
| | Protein C14orf159, mitochondrial precursor |
| | (UNQ2439/PRO5000)¥ gb\|AAH10614.1\| |
| | C14orf159 protein [Human]¥ gb\|AAH65558.1\|C14orf159 |
| | protein [Human] |
| | gb\|AAQ88880.1\|PFTL2439 |
| | [Human]¥ ref\|NP_079228.3\| |
| | chromosome 14 open reading frame 159 [Human] |
| | emb\|CAD61880.1\|unnamed protein product [Human] |
| | emb\|CAG33570.1\|C14orf159 [Human] |

TABLE 5-2

| | |
|---|---|
| | emb\|CAD97934.1\|hypothetical protein [Human] |
| | dbj\|BAB14102.1\|unnamed protein product |
| | [Human]¥ sp\|Q7Z3D6\|CN159_HUMAN Protein C14orf159, |
| | mitochondrial precursor |
| | (UNQ2439/PRO5000)¥ gb\|AAH10614.1\| |
| | C14orf159 protein [Human]¥ gb\|AAH65558.1\|C14orf159 |
| | protein [Human] |
| FLJ12514 | dbj\|BAB14108.1\|unnamed protein product [Human] |
| | dbj\|BAB14327.1\|unnamed protein product |
| | [Human]¥ gb\|AAH02931.1\|CCR4-NOT transcription |
| | complex, subunit 10 [Human]¥ |
| | ref\|NP_056257.1\|CCR4-NOT |
| | transcription complex, subunit 10 [Human] |
| | dbj\|BAB14478.1\|unnamed protein product [Human] |
| | ref\|NP_705813.2\|CCR4-NOT transcription |
| | complex, subunit 10 |
| | [Mouse]¥ gb\|AAH39183.1\|CCR4-NOT transcription complex, |
| | subunit 10 [Mouse]¥ dbj\|BAC26299.1\|unnamed protein |
| | product [Mouse] |
| | gb\|AAH83782.1\|Similar to RIKEN cDNA 2600001P13 |
| | [Rat]¥ ref\|NP_001007004.1\|similar |
| | to RIKEN cDNA 2600001P13 |
| | [Rat] |
| | dbj\|BAB14108.1\|unnamed protein product [Human] |
| FLJ14583 | ref\|NP_057241.2\|trophinin isoform 2 |
| | [Human]¥ ref\|NP_808224.1\|trophinin isoform 2 |
| | [Human]¥ dbj\|BAB55149.1\|unnamed protein |
| | product [Human] |
| | gb\|AAK30171.1\|magphinin β [Human] |
| | dbj\|BAA83066.3\|KIAA1114 protein [Human] |
| | ref\|NP_808223.1\|trophinin isoform 1 |
| | [Human]¥ gb\|AAH26914.1\|Trophinin, isoform 1 [Human] |
| | gb\|AAK30170.1\|magphinin α [Human] |
| | ref\|NP_057241.2\|trophinin isoform 2 |
| | [Human]¥ ref\|NP_808224.1\|trophinin isoform 2 |
| | [Human]¥ dbj\|BAB55149.1\|unnamed |
| | protein product [Human] |
| FLJ31146 | gb\|AAH05091.1\|FLJ22313 protein |
| | [Human]¥ ref\|NP_071768.2\| |
| | hypothetical protein LOC64224 [Human]¥ gb\|AAH20264.1\| |
| | Hypothetical protein FLJ22313 [Human] |
| | gb\|EAL23973.1\|hypothetical protein FLJ22313 [Human] |
| | gb\|AAH29691.1\|RIKEN cDNA 5031400M07 [Mouse] |
| | ref\|NP_065611.1\|hypothetical protein LOC80517 |
| | [Mouse]¥ gb\|AAH43693.1\|RIKEN cDNA 5031400M07 |
| | [Mouse]¥ dbj\|BAA95064.1\|unnamed protein |
| | product [Mouse] |
| | dbj\|BAB31889.1\|unnamed protein product [Mouse] |
| | gb\|AAH05091.1\|FLJ22313 protein |
| | [Human]¥ ref\|NP_071768.2\| |
| | hypothetical protein LOC64224 [Human]¥ gb\|AAH20264.1\| |
| | Hypothetical protein FLJ22313 [Human] |

### Nucleic Acid Analysis 2

### Homology Analysis 2 by BLASTX

The calculation program used was blastall 2.2.6. The target databases used were swiss-prot:146720 (2004.03.29), (Refseq)hs:21170 (2004.05.06), (Refseq)mouse:17089 (2004.05.06), and (Refseq)rat:4893 (2004.05.06). The cutoff value was established at 1.00E-05. The following data were processed by filtering:

### For Swiss-Prot:

Having a definition beginning with “ALU SUBFAMILY”

Having a definition beginning with “Alu subfamily”

Having a definition beginning with “!!!! ALU SUBFAMILY”

Having a definition beginning with “B-CELL GROWTH FACTOR PRECURSOR”

Having a definition including “NRK2”

Having a definition beginning with “PROLINE-RICH”

Having a definition beginning with “GLYCINE-RICH”

Having a definition beginning with “EXTENSIN PRECURSOR”

Having a definition beginning with “COLLAGEN”

Having a definition beginning with “100 KD”

Having a definition beginning with “RETROVIRUS-RELATED POL POLYPROTEIN”

Having a definition beginning with “CUTICLE COLLAGEN”

Having a definition beginning with “HYPOTHETICAL”

Having a definition beginning with “Hypothetical”

Having a definition beginning with “SALIVARY PROLINE-RICH PROTEIN”

Having a definition beginning with “IMMEDIATE-EARLY PROTEIN”

Having the accession No “P49646”

### For Ref-Seq:

Having a definition beginning with “hypothetical protein FLJ”

Having a definition beginning with “KIAA”

Having a definition beginning with “hypothetical protein DKFZ”

Having a definition beginning with “DKFZ”

Having a definition beginning with “RIKEN cDNA”

Having a definition beginning with “hypothetical protein MGC”

Having a definition as “hypothetical protein”

Having a definition beginning with “hypothetical protein PP”

Having a definition as “neuronal thread protein”

Having a definition beginning with “clone FLB”

Having a definition beginning with “hypothetical protein PRO”

Having a definition as “PRO0483 protein”

Having a definition including “MNC”

Having a definition including “MOST-1”

Having a definition beginning with “similar to”

Having a definition including “TPR gene on Y”

Having a definition beginning with “HSPC”

Having a definition beginning with “CGI-”

ReFSeq sequence composed of self only (information referenced from LL_tmpl)

The annotation information obtained by this analysis is shown in Tables 6-1 to 6-2.

TABLE 6-1

| FLJ No. | Accession No. and definition | Key words |
|---|---|---|
| FLJ10368 | Q9NUX5 Protection of telomeres 1 (hPot1) | Alternative |
| | (POT1-like telomere end-binding protein) | splicing; |
| | Q95K48 Protection of telomeres 1 (POT1-like | Chromosomal |
| | telomere end-binding protein) | protein; DNA-binding; |
| | Q91WC1 Protection of telomeres 1 (mPot1) | Nuclear |
| | (POT1-like telomere end-binding protein) | protein; |
| | Q9NUX5 Protection of telomeres 1 (hPot1) | Telomere. |
| | (POT1-like telomere end-binding protein) | |
| | NP_056265.1 Protection of telomeres 1 [Human] | |
| | NP_598692.1 POT1-like telomere end-binding | |
| | protein [Mouse] | |
| | NP_056265.1 protection of telomeres 1 [Human] | |
| FLJ12389 | Q9Z3R3 Acetoacetyl-coenzyme A synthetase (EC | Acetylation; |
| | 6.2.1.16) (Acetoacetate-CoA ligase 1) (Acyl- | Complete |
| | activating enzyme 1) | proteome; |
| | Q9NR19 Acetyl-coenzyme A synthetase, | Ligase. |
| | cytoplasmic (EC6.2.1.1) (Acetate-CoA ligase) | |
| | (Acyl-activating enzyme) (Acetyl-CoA | |
| | synthetase) (ACS) (AceCS) | |
| | Q9RRL7 Acetyl-coenzyme A synthetase (EC | |
| | 6.2.1.1) (Acetate-CoA ligase) (Acyl- | |
| | activating enzyme) | |
| | Q9Z3R3 Acetoacetyl-coenzyme A synthetase (EC | |
| | 6.2.1.16) (Acetoacetate-CoA ligase 1) (Acyl- | |
| | activating enzyme 1) | |
| | NP_076417.2 acetoacetyl-CoA synthetase | |
| | [Human] | |
| | NP_084486.1 acetoacetyl-CoA synthetase | |
| | [Mouse] | |
| | NP_075592.1 acetoacetyl-CoA synthetase [Rat] | |
| | NP_076417.2 acetoacetyl-CoA synthetase | |
| | [Human] | |
| FLJ12435 | NP_065385.2 pre-B-cell leukemia transcription | |
| | factor interacting protein 1 [Human] | |
| | NP_666243.1 pre-B-cell leukemia transcription | |
| | factor interacting protein 1 [Mouse] | |
| | NP_065790.1 cell cycle progression 1 [Human] | |
| | NP_065385.2 pre-B-cell leukemia transcription | |
| | factor interacting protein 1 [Human] | |
| FLJ12502 | Q9H8Y9 Protein C14orf159, mitochondrial | Alternative |
| | precursor (UNQ2439/PRO5000) | splicing; |
| | Q8BH86 Protein C14orf159 homolog, | Mitochondrion; |
| | mitochondrial precursor | Transit |
| | Q9H8Y9 Protein C14orf159, mitochondrial | peptide. |
| | precursor (UNQ2439/PRO5000) | |
| | NP_079228.3 chromosome 14 open reading frame | |
| | 159 [Human] | |
| | NP_079228.3 chromosome 14 open reading frame | |
| | 159 [Human] | |
| FLJ12514 | NP_056257.1 CCR4-NOT transcription complex, | |
| | subunit 10 [Human] | |
| | NP_705813.2 CCR4-NOT transcription complex, | |
| | subunit 10 [Mouse] | |
| | NP_056257.1 CCR4-NOT transcription complex, | |
| | subunit 10 [Human] | |

TABLE 6-2

| | | |
|---|---|---|
| FLJ14583 | Q12816 Trophinin (MAGE-D3 antigen) | Antigen; Cell |
| | Q9UNF1 Melanoma-associated antigen D2 (MAGE- | adhesion; |
| | D2 antigen) (MAGE-D) (Breast cancer | Repeat; |
| | associated gene 1 protein) (BCG-1) (11B6) | Alternative |
| | (Hepatocellular carcinoma associated protein | splicing; |
| | JCL-1) | Multigene |
| | Q9QYH6 Melanoma-associated antigen D1 | family; Polymorphism. |
| | (MAGE-D1 antigen) (Neurotrophin receptor- | |
| | interacting MAGE homolog) (Dlxin-1) | |
| | Q12816 Trophinin (MAGE-D3 antigen) | |
| | NP_808224.1 trophinin isoform 2 [Human] | |
| | NP_057241.2 trophinin isoform 2 [Human] | |
| | NP_808223.1 trophinin isoform 1 [Human] | |
| | NP_808224.1 trophinin isoform 2 [Human] | |
| FLJ31146 | Q9JJK5 Homocysteine-responsive endoplasmic | Endoplasmic |
| | reticulum-resident ubiquitin-like domain | reticulum; |
| | member 1 protein | Transmembrane; |
| | Q15011 Homocysteine-responsive endoplasmic | Unfolded |
| | reticulum-resident ubiquitin-like domain | protein |
| | member 1 protein (Methyl methanesulfonate | response; |
| | (MMF)-inducible fragment protein 1) | Alternative |
| | Q6B4Z3 Ubiquitously transcribed Y chromosome | splicing; |
| | tetratricopeptide repeat protein | Nuclear |
| | (Ubiquitously transcribed TPR protein on the | protein; |
| | Y chromosome) | Repeat; TPR |
| | Q9JJK5 Homocysteine-responsive endoplasmic | repeat. |
| | reticulum-resident ubiquitin-like domain | |
| | member 1 protein | |
| | NP_071726.1 homocysteine-inducible, | |
| | endoplasmic reticulum stress-inducible, | |
| | ubiquitin-like domain member 1 [Mouse] | |
| | NP_445975.1 homocysteine-inducible, | |
| | endoplasmic reticulum stress-inducible, | |
| | ubiquitin-like domain member 1 [Rat] | |
| | NP_055500.1 homocysteine-inducible, | |
| | endoplasmic reticulum stress-inducible, | |
| | ubiquitin-like domain member 1 isoform 1 | |
| | [Human] | |
| | NP_071768.2 hypothetical protein FLJ22313 | |
| | [Human] | |

Other examples of possible diseases or conditions are the diseases or conditions registered with OMIM. These diseases or conditions can easily be searched by, for example, inputting H-Inv ID numbers or H-Inv cluster ID numbers in H-Inv DB. The chromosomes and gene loci where the target genes for bioactive substances in this application are present, and OMIM information on orphan diseases expected to be associated with these genes, are shown in Tables 7-1 to 7-4.

TABLE 7-1

| FLJ | | Genome | Gene | OMIM disease information |
|---|---|---|---|---|
| No. | Chromosome | locus | Cluster | (OMIM Co-localized orphan disease) |
| FLJ10368 | 7 | 124017816-124091753, | chr7 − | OMIM603678: DEAFNESS, AUTOSOMAL |
| | | minus | 1452 | RECESSIVE 14; DFNB14 |
| | | (genome | | OMIM60302.0: DEAFNESS, NEUROSENSORY, |
| | | align | | AUTOSOMAL RECESSIVE 17; DFNB17 |
| | | coverage | | OMIM603511: MUSCULAR DYSTROPHY, |
| | | of cDNA | | LIMB-GIRDLE, TYPE 1D; LGMD1D |
| | | length: | | OMIM607454: SPINOCEREBELLAR ATAXIA |
| | | 99.6%) | | 21; SCA21 |
| FLJ12389 | 12 | 123903083-123980960, | chr12 + 1640 | OMIM121400: CORNEA PLANA 1; CNA1 |
| | | plus | | OMIM605583: DEAFNESS, AUTOSOMAL |
| | | (genome | | DOMINANT NONSYNDROMIC SENSORINEURAL |
| | | align | | 25; DFNA25 |
| | | coverage | | |
| | | of cDNA | | |
| | | length: | | |
| | | 99.3%) | | |
| FLJ12435 | 1 | 152133344-152145391, | chr1 − | OMIM605803: DERMATITIS, ATOPIC, 2; |
| | | minus | 2003 | ATOD2 |
| | | (genome | | OMIM605549: CONE-ROD DYSTROPHY 8; |
| | | align | | CORD8 |
| | | coverage | | OMIM601412: DEAFNESS, AUTOSOMAL |
| | | of cDNA | | DOMINANT NONSYNDROMIC SENSORINEURAL |
| | | length: | | 7; DFNA7 |
| | | 99.1%) | | OMIM174000: MEDULLARY CYSTIC KIDNEY |
| | | | | DISEASE 1; MCKD1 |
| | | | | OMIM603935: PSORIASIS |
| | | | | SUSCEPTIBILITY 4; PSORS4 |
| | | | | OMIM605642: THYROID CARCINOMA, |
| | | | | PAPILLARY, WITH PAPILLARY RENAL |
| | | | | NEOPLASIA |

TABLE 7-2

| | | | | |
|---|---|---|---|---|
| FLJ12502 | 14 | 89570827-89681737, | chr14 + 855 | OMIM213600: BASAL GANGLIA |
| | | plus | | CALCIFICATION, IDIOPATHIC, 1; IBGC1 |
| | | (genome | | OMIM164210: HEMIFACIAL MICROSOMIA; |
| | | align | | HFM |
| | | coverage of | | OMIM270100: SITUS INVERSUS VISCERUM |
| | | cDNA | | OMIM138800: GOITER, MULTINODULAR 1; |
| | | length: | | MNG1 |
| | | 99.6%) | | OMIM160500: MYOPATHY, DISTAL 1; MPD1 |
| | | | | OMIM252930: MUCOPOLYSACCHARIDOSIS |
| | | | | TYPE IIIC |
| | | | | OMIM276900: USHER SYNDROME, TYPE IA; |
| | | | | USH1A |
| FLJ12514 | 3 | 32698624-32786972 | chr3 + 381 | OMIM604400: ARRHYTHMOGENIC RIGHT |
| | | | | VENTRICULAR DYSPLASIA, FAMILIAL, 5; |
| | | | | ARVD5 |
| | | | | OMIM601154: CARDIOMYOPATHY, DILATED, |
| | | | | 1E; CMD1E |
| | | | | OMIM601869: DEAFNESS, AUTOSOMAL |
| | | | | RECESSIVE 15; DFNB15 |
| | | | | OMIM608088: NEUROPATHY, HEREDITARY |
| | | | | SENSORY, TYPE I, WITH COUGH AND |
| | | | | GASTROESOPHAGEAL REFLUX |
| | | | | OMIM182280: SMALL CELL CANCER OF THE |
| | | | | LUNG |
| | | | | OMIM276905: USHER SYNDROME, TYPE |
| | | | | IIB; USH2B |

TABLE 7-3

| | | | | |
|---|---|---|---|---|
| FLJ14583 | X | 53914048-53924653, | chrX + 442 | OMIM301830: ARTHROGRYPOSIS MULTIPLEX |
| | | plus | | CONGENITA, DISTAL, X-LINKED |
| | | (genome | | OMIM300404: BRAIN ANOMALIES, |
| | | align | | RETARDATION, ECTODERMAL DYSPLASIA, |
| | | coverage | | SKELETAL MALFORMATIONS, HIRSCHSPRUNG |
| | | of cDNA | | DISEASE, EAR/EYE ANOMALIES, CLEFT |
| | | length: | | PALATE/CRYPTORCHIDISM, AND KIDNEY |
| | | 100%) | | DYSPLASIA/HYPOPLASIA |
| | | | | OMIM300220: CHORIOATHETOSIS WITH MENTAL |
| | | | | RETARDATION AND ABNORMAL BEHAVIOR |
| | | | | OMIM302802: CHARCOT-MARIE-TOOTH |
| | | | | DISEASE, X-LINKED RECESSIVE, 3; CMTX3 |
| | | | | OMIM300331: THROMBOCYTOSIS, FAMILIAL X- |
| | | | | LINKED |
| | | | | OMIM300268: ANGIONEUROTIC EDEMA, |
| | | | | HEREDITARY, WITH NORMAL C1-INHIBITOR |
| | | | | CONCENTRATION AND FUNCTION |
| | | | | OMIM300136: DIABETES MELLITUS, INSULIN- |
| | | | | DEPENDENT, X-LINKED, SUSCEPTIBILITY TO |
| | | | | OMIM300290: INTRAUTERINE GROWTH |
| | | | | RETARDATION, METAPHYSEAL DYSPLASIA, |
| | | | | ADRENAL HYPOPLASIA CONGENITA, AND |
| | | | | GENITAL ANOMALIES |
| | | | | OMIM300345: MICROPHTHALMIA, |
| | | | | COLOBOMATOUS, 1; MOPCB1 |
| | | | | OMIM309530: MENTAL RETARDATION, X- |
| | | | | LINKED 1; MRX1 |
| | | | | OMIM300062: MENTAL RETARDATION, X- |
| | | | | LINKED 14; MRX14 |
| | | | | OMIM300047: MENTAL RETARDATION, X- |
| | | | | LINKED 20; MRX20 |
| | | | | OMIM300115: MENTAL RETARDATION, X- |
| | | | | LINKED 50; MRX50 |
| | | | | OMIM300210: MENTAL RETARDATION, X- |
| | | | | LINKED 58; MRX58 |
| | | | | OMIM309545: MENTAL RETARDATION, X- |
| | | | | LINKED NONSPECIFIC, WITH APHASIA; MRXA |
| | | | | OMIM300218: MENTAL RETARDATION, X- |
| | | | | LINKED, SYNDROMIC 7; MRXS7 |
| | | | | OMIM300263: SIDERIUS X-LINKED MENTAL |
| | | | | RETARDATION SYNDROME |
| | | | | OMIM311400: PAINE SYNDROME |
| | | | | OMIM300373: OSTEOPATHIA STRIATA WITH |
| | | | | CRANIAL SCLEROSIS; OSCS |
| | | | | OMIM309610: PRIETO X-LINKED MENTAL |
| | | | | RETARDATION SYNDROME; PRS |
| | | | | OMIM309500: RENPENNING SYNDROME 1; |
| | | | | RENS1 |
| | | | | OMIM300258: ROIFMAN SYNDROME |
| | | | | OMIM309585: WILSON-TURNER X-LINKED |
| | | | | MENTAL RETARDATION SYNDROME; WTS |

TABLE 7-4

| | | | | |
|---|---|---|---|---|
| FLJ31146 | 7 | 35414680-35475974, | chr7 − 422 | OMIM141400: HEMIFACIAL MICROSOMIA |
| | | minus | | WITH RADIAL DEFECTS |
| | | (genome | | OMIM607454: SPINOCEREBELLAR ATAXIA |
| | | align | | 21; SCA21 |
| | | coverage of | | |
| | | cDNA | | |
| | | length: | | |
| | | 99.3%) | | |

Other possible diseases or conditions are diseases or conditions accompanied by abnormalities at expression sites of target gene Y, or in tissues from which the library for target gene Y is derived. The expression sites and tissues can easily be searched by, for example, inputting H-Inv cDNA ID numbers or H-Inv locus ID numbers in H-Inv DB, whereby those skilled in the art are able to postulate the diseases or conditions.

For example, some of target gene Y are expressed at the sites shown below.

FLJ10368-derived protein can be expressed in cerebrum, cerebellum, corpus callosum, glia, retina, spleen, thymus, uterus, placenta, testis, heart, muscle, colon, small intestine, liver, lung, kidney, mammary gland, pituitary gland, thyroid gland and the like.

FLJ12389-derived protein can be expressed in brain stem, cerebellum, blood, esophagus, skin, ovary, prostate, testis, heart, muscle, colon, stomach, liver, lung, adrenal gland, mammary gland, salivary gland and the like.

FLJ12435-derived protein can be expressed in brain stem, cerebrum, cerebellum, corpus callosum, glia, retina, spinal cord, lymph node, spleen, thymus, esophagus, skin, uterus, ovary, placenta, prostate, testis, muscle, stomach, bladder, adrenal gland, pituitary gland, thyroid gland and the like.

FLJ12502-derived protein can be expressed in brain stem, cerebellum, lymph nodes, blood, spleen, prostate, testis, heart, muscle, colon, stomach, liver, lung, kidney, adrenal gland, pancreas, pituitary gland and the like.

FLJ12514-derived protein can be expressed in brain stem, corpus callosum, glia, retina, lymph node, spleen, thymus, skin, placenta, prostate, testis, muscle, colon, small intestine, liver, adrenal gland, thyroid gland and the like.

FLJ14583-derived protein can be expressed in brain stem, cerebrum, cerebellum, corpus callosum, glia, is spinal cord, bone marrow, bone, skin, ovary, placenta, prostate, testis, heart, muscle, small intestine, liver, lung, mammary gland, pancreas, pituitary gland, salivary gland and the like.

FLJ31146-derived protein can be expressed in brain stem, cerebrum, eye, spleen, thymus, bone, skin, uterus, placenta, prostate, testis, muscle, colon, lung, adrenal gland, mammary gland and the like.

Still other examples of possible diseases or conditions are diseases or conditions mediated by genes that are homologous to target gene Y or a gene downstream thereof. Those skilled in the art are able to postulate such diseases or conditions by identifying homologous genes by homology search, and then extensively investigating the diseases or conditions involved by the homologous genes by a commonly known method.

The target proteins and target genes of the present invention are useful for, for example, the development of drugs for specified diseases or conditions, or the development of investigational reagents for the diseases or conditions, and the like.

### 2. Screening Methods and Products Obtained by the Methods

The present invention provides screening methods for bioactive substances, each of which comprises determining whether or not a test substance is capable of regulating the expression or function of a target protein for the bioactive substance or a gene that encodes the protein (hereinafter sometimes referred to as “target protein Y” or “target gene Y” as required), and a product thereof. The screening methods of the present invention can be roughly divided into two types, from the viewpoint of the kind of bioactive substance screened: screening methods for substances capable of regulating an action associated with bioactive substance X, and screening methods for substances capable of regulating a function associated with target protein Y. The screening methods of the present invention can also be performed in vitro, in vivo or in silico. A substance capable of regulating the expression of target protein Y obtained by the screening method of the present invention has the same definition as a substance capable of regulating the level of target protein Y; it can be a substance capable of altering the abundance of target protein Y in a specified tissue or cell, or the abundance of target protein Y in a specified intracellular site. Accordingly, examples of the substances capable of regulating the expression of target protein Y include not only substances capable of regulating the biosynthesis of target protein Y from target gene Y, but also substances capable of regulating the intracellular localization of target protein Y, substances capable of regulating in vivo kinetics of target protein Y, and substances capable of regulating the metabolism (e.g., metabolic synthesis and decomposition) of target protein Y.

The individual screening methods are hereinafter described in detail.

2.1. Screening Methods for Substances Capable of Regulating an Action Associated with Bioactive Substance X (Screening Method I)

The present invention provides screening methods for substances capable of regulating an action associated with bioactive substance X, each of which comprises determining whether or not a test substance is capable of regulating the expression or function of target protein Y.

The screening methods of this type are generically referred to as “screening method I” as required.

Screening method I can be roughly divided into two types: a screening method for a substance capable of regulating an action associated with bioactive substance X, which comprises determining whether or not a test substance is capable of regulating the expression or function of target protein Y, and selecting a test substance capable of regulating the expression or function of target protein Y (screening method Ia), and a screening method for a substance capable of regulating an action associated with bioactive substance X (particularly an action associated with a known target molecule), which comprises determining whether or not a test substance is capable of regulating the expression or function of target protein Y, and selecting a test substance that is incapable of regulating the expression or function of target protein Y (screening method Ib). Screening method Ia can be useful for the development of regulators of diseases or conditions associated with bioactive substance X and the like. Screening method Ib can be useful for the development of drugs capable of regulating an action associated with a known target molecule, and showing decreased adverse effects of bioactive substance X and the like.

2.1.1. Screening Method for Substances Capable of Regulating an Action Associated with Bioactive Substance X, Which Comprises Selecting a Test Substance Capable of Regulating the Expression or Function of Target Protein Y (Screening Method Ia)

The present invention provides a screening method for substances capable of regulating an action associated with bioactive substance X, which comprises determining whether or not a test substance is capable of regulating the expression or function of target protein Y, and selecting a test substance capable of regulating the expression or function of target protein Y.

The test substance subjected to this screening method may be any known compound or new compound; examples include nucleic acids, carbohydrates, lipids, proteins, peptides, organic small compounds, compound libraries prepared using combinatorial chemistry technique, random peptide libraries prepared by solid phase synthesis or the phage display method, or natural components derived from microorganisms, animals, plants, marine organisms and the like, and the like. The test substance may be a labeled supply or a non-labeled supply, or a mixture of a labeled supply and a non-labeled supply mixed in a specified ratio. The labeling substance is the same as described above.

In one embodiment, screening method Ia comprises the following steps (a), (b) and (c):

(a) a step for bringing the test substance into contact with target protein Y;
(b) a step for measuring the functional level of the protein in the presence of the test substance, and comparing this functional level with the functional level of the protein in the absence of the test substance;
(c) a step for selecting a test substance that alters the functional level of the protein on the basis of the result of the comparison in step (b) above.

The methodology comprising the above-described steps (a) to (c) is referred to as “methodology I” as required.

In step (a) of methodology I, a test substance is brought into contact with target protein Y. Contact of the test substance with the protein can be performed by contact of isolated target protein Y and the test substance in solution, or contact of cells or tissue capable of expressing target protein Y and the test substance.

Target protein Y can be prepared by a method known per se. For example, target protein Y can be isolated and purified from the above-described expression tissue. However, to prepare target protein Y quickly, easily, and in large amounts, and to prepare human target protein Y, it is preferable to prepare a recombinant protein by gene recombination technology. The recombinant protein may be prepared using a cell system or a cell-free system.

The cells capable of expressing target protein Y can be any cells that express target protein Y; examples include cells derived from the tissue in which target protein Y is expressed, cells transformed with target protein Y expression vector and the like. Those skilled in the art are able to easily identify or prepare these cells; useful cells include primary culture cells, cell lines derivatively prepared from the primary culture cells, commercially available cell lines, cell lines available from cell banks, and the like. As the tissue capable of expressing target protein Y, the above-described expression tissues can be used.

In step (b) of methodology I, the functional level of the protein in the presence of the test substance is measured. A measurement of the functional level can be performed according to the kind of protein by a method known per se. For example, provided that target protein Y is a transcription factor, a substance that regulates a function associated with target protein Y can be screened by performing a reporter assay using target protein Y and a transcription regulatory region to which it binds.

Provided that target protein Y is an enzyme, the functional level can also be measured on the basis of a change in the catalytic activity of the enzyme. The catalytic activity of the enzyme can be measured by a method known per se using a substrate, coenzyme and the like chosen as appropriate according to the kind of enzyme.

Furthermore, provided that target protein Y is a membrane protein (e.g., receptor, transporter), the functional level can be measured on the basis of a change in a function of the membrane protein. For example, provided that target protein Y is a receptor, a screening method of the present invention can be performed on the basis of an intracellular event mediated by the receptor (e.g., inositol phospholipid production, intracellular pH change, intracellular behavior of ions such as calcium ion and chlorine ion). Provided that target protein Y is a transporter, a screening method of the present invention can be performed on the basis of a change in the intracellular concentration of a substrate for the transporter.

The functional level may also be measured on the basis of the functional level of target protein Y to each isoform (e.g., splicing variant) or the isoform-isoform functional level ratio, rather than on the basis of the total functional level of target protein Y.

Next, the functional level of target protein Y in the presence of the test substance is compared with the functional level of target protein Y in the absence of the test substance. This comparison of functional level is preferably performed on the basis of the presence or absence of a significant difference. Although the functional level of target protein Y in the absence of the test substance may be measured prior to, or simultaneously with, the measurement of the functional level of target protein Y in the presence of the test substance, it is preferable, from the viewpoint of experimental accuracy and reproducibility, that the functional level be measured simultaneously.

In step (c) of methodology I, a test substance that alters the functional level of the protein is selected. The test substance that alters the functional level of the protein is capable of promoting or suppressing a function of target protein Y. The test substance thus selected can be useful for the regulation of a disease or condition associated with bioactive substance X.

Methodology I may be performed not only in the presence of target protein Y but also with a coupling factor thereof. For example, when target protein Y inhibitory factor is used in combination as the coupling factor of target protein Y, a substance that interferes with the interaction between target protein Y and the coupling factor is considered to be capable of promoting a function of target protein Y. When target protein Y activation factor is used in combination as the coupling factor for target protein Y, a substance that interferes with the interaction between target protein Y and the coupling factor is considered to be capable of suppressing a function of target protein Y. Hence, it is also beneficial to perform methodology I in the presence of a coupling factor of target protein Y.

In another embodiment, screening method Ia comprises the following steps (a), (b) and (c):

(a) a step for bringing the test substance and cells enabling a measurement of the expression of target protein Y or a gene that encodes the protein into contact with each other;
(b) a step for measuring the expression level in the cells in contact with the test substance, and comparing this expression level with the expression level in control cells not in contact with the test substance;
(c) a step for selecting a test substance that regulates the expression level on the basis of the result of the comparison in step (b) above.

The methodology comprising the above-described steps (a) to (c) is referred to as “methodology II” as required.

In step (a) of methodology II, a test substance is brought into contact with cells enabling a measurement of the expression of target protein Y. Contact of the test substance with the cells enabling a measurement of the expression of target protein Y can be performed in culture medium.

“Cells enabling a measurement of the expression of target protein Y or a gene that encodes the protein (referred to as “target gene Y” as required)” refers to cells enabling a direct or indirect evaluation of the expression level of a product of target gene Y, for example, a transcription product or translation product (i.e., protein). The cells enabling a direct evaluation of the expression level of a product of target gene Y can be cells capable of naturally expressing target gene Y, whereas the cells enabling an indirect evaluation of the expression level of a product of target gene Y can be cells enabling a reporter assay on target gene Y transcription regulatory region.

The cells capable of naturally expressing target gene Y can be any cells that potentially express target gene Y; examples include cells showing constitutive expression of target gene Y, cells that express target gene Y under inductive conditions (e.g., drug treatment) and the like. Those skilled in the art are able to easily identify these cells; useful cells include primary culture cells, cell lines derivatively prepared from the primary culture cells, commercially available cell lines, cell lines available from cell banks, and the like.

The cells enabling a reporter assay on target gene Y transcription regulatory region are cells incorporating target gene Y transcription regulatory region and a reporter gene functionally linked to the region. The target gene Y transcription regulatory region and reporter gene are inserted in an expression vector.

The target gene Y transcription regulatory region may be any region enabling the control of the expression of target gene Y; examples include a region from the transcription initiation point to about 2 kbp upstream thereof, and a region consisting of a base sequence wherein one or more bases are deleted, substituted or added in the base sequence of the region, and that is capable of controlling the transcription of target gene Y, and the like.

The reporter gene may be any gene that encodes a detectable protein or enzyme; examples include the GFP (green fluorescent protein) gene, GUS (β-glucuronidase) gene, LUS (luciferase) gene, CAT (chloramphenicol acetyltransferase) gene and the like.

The cells transfected with target gene Y transcription regulatory region and a reporter gene functionally linked to the region are not subject to limitation, as long as they enable an evaluation of target gene Y transcription regulatory function, that is, as long as they enable a quantitative analysis of the expression level of the reporter gene. However, the cells transfected are preferably cells capable of naturally expressing target gene Y because they are considered to express a physiological transcription regulatory factor for target gene Y, and to be more appropriate for the evaluation of the regulation of the expression of target gene Y.

The culture medium in which a test substance and cells enabling a measurement of the expression of target gene Y are brought into contact with each other is chosen as appropriate according to the kind of cells used and the like; examples include minimal essential medium (MEM) containing about 5 to 20% fetal bovine serum, Dulbecco's modified minimal essential medium (DMEM), RPMI1640 medium, 199 medium and the like. Culture conditions are also determined as appropriate according to the kind of cells used and the like; for example, the pH of the medium is about 6 to about 8, culture temperature is normally about 30 to about 40° C., and culture time is about 12 to about 72 hours.

In step (b) of methodology II, first, the expression level of target gene Y in the cells in contact with the test substance is measured. This measurement of expression level can be performed by a method known per se in view of the kind of cells used and the like.

For example, when cells capable of naturally expressing target gene Y are used as the cells enabling a measurement of the expression of target gene Y, the expression level can be measured by a method known per se with a product of target gene Y, for example, a transcription product or translation product, as the subject. For example, the expression level of a transcription product can be measured by preparing total RNA from the cells, and performing RT-PCR, Northern blotting and the like. The expression level of a translation product can be measured by preparing an extract from the cells, and performing an immunological technique. Useful immunological techniques include radioimmunoassay (RIA) method, ELISA method (Methods in Enzymol. 70: 419-439 (1980)), fluorescent antibody technique and the like.

When cells enabling a reporter assay on target gene Y transcription regulatory region are used as the cells enabling a measurement of the expression of target gene Y, the expression level can be measured on the basis of the signal intensity of the reporter.

The expression level may also be measured on the basis of the expression level of target gene Y to each isoform (e.g., splicing variant) or the isoform-isoform expression ratio, rather than on the basis of the total expression level of target gene Y.

Furthermore, when target gene Y is a gene of a factor that is localized intracellularly, the expression level can also be measured based on the intracellular localization. The level of target protein Y localized in a specified intracellular organelle can be measured by a method known per se. The level can be measured, for example, by introducing target gene Y fused with a gene encoding a fluorescence protein such as GFP gene into an appropriate cell and cultivating the cell in a culture medium in the presence of a test substance, then observing the fluorescence signal in the specified intracellular organelle with a confocal microscope, and comparing the fluorescence signal with that in the organ in the absence of the test substance. The level of target protein Y localized in a specified intracellular organelle can also be measured by immunostaining using an antibody against target protein Y.

Next, the expression level of target gene Y in the cells in contact with the test substance is compared with the expression level of target gene Y in control cells not in contact with the test substance. This comparison of expression level is preferably performed on the basis of the presence or absence of a significant difference. Although the expression level of target gene Y in the control cells not in contact with the test substance may be measured prior to, or simultaneously with, the measurement of the expression level of target gene Y in the cells in contact with the test substance, it is preferable, from the viewpoint of experimental accuracy and reproducibility, that the expression level be measured simultaneously.

In step (c) of methodology II, a test substance that regulates the expression level of target gene Y is selected. The regulation of the expression level of target gene Y can be the promotion or suppression of the expression level. The test substance thus selected can be useful for the regulation of an action associated with bioactive substance X.

In another embodiment, screening method Ia comprises the following steps (a), (b) and (c):

(a) a step for bringing the test substance into contact with target protein Y;
(b) a step for measuring the ability of the test substance to bind to the protein;
(c) a step for selecting a test substance capable of binding to the protein on the basis of the results of step (b) above.

The methodology comprising the above-described steps (a) to (c) is referred to as “methodology III” as required.

In step (a) of methodology III, a test substance is brought into contact with target protein Y. Contact of the test substance with the protein can be performed by mixing the test substance and the protein in solution.

Target protein Y can be prepared by a method known per se. For example, target protein Y can be isolated and purified from the above-described target-gene Y expression tissue. However, to prepare target protein Y quickly, easily, and in large amounts, and to prepare human target protein Y, it is preferable to prepare a recombinant protein by gene recombination technology. The recombinant protein may be prepared using a cell system or a cell-free system.

In step (b) of methodology III, the ability of the test substance to bind to the protein is measured. “A binding ability” measured may be any one that enables an evaluation of the binding of the protein and the test substance; examples include binding amount, binding strength (including parameters such as affinity constant, binding rate constant, and dissociation rate constant), and binding mode (including dose-dependent binding).

A measurement of the binding ability can be performed by, for example, the SEC/MS method (size exclusion chromatography/mass spectrometry) (see Moy, F. J. et al., Anal. Chem., 2001, 73, 571-581). The SEC/MS method comprises (1) a step for adding a mixed multiplexed compound sample to the purified protein, and then separating the free compound and the protein by SEC, and (2) an analytical step for identifying the bound compound contained in the protein fraction by MS. The SEC/MS method is advantageous in that the binding ability can be analyzed while both the protein and the test substance are in non-modified and non-immobilized state. In the SEC/MS method, not only the ability of the test substance to bind to the protein, but also the dose dependency of the test substance in the binding to the protein and the like can be measured simultaneously.

A measurement of the binding ability can also be performed using a means for measurement based on surface plasmon resonance, for example, Biacore. Using Biacore, the binding and dissociation of a test substance to a protein immobilized on a chip are measured, and the measured values are compared with those obtained when a solution not containing the test substance is loaded on the chip. Subsequently, a test substance capable of binding to the protein is selected on the basis of the result for the binding and dissociation rate or binding amount. Biacore also enables simultaneous measurements of binding strength (e.g., Kd value) and the like, in addition to the ability of a test substance to bind to a protein.

Other methods for measuring the binding ability include, for example, SPR-based methods or optical methods such as the quartz crystal microbalance (QCM) method, the dual polarization interferometer (DPI) method, and the coupled waveguide plasmon resonance method, immunoprecipitation, isothermal titration and differential scanning calorimetry, capillary electrophoresis, energy transfer, fluorescent analytical methods such as fluorescent correlation analysis, and structural analytical methods such as X-ray crystallography and nuclear magnetic resonance (NMR).

In measuring the binding ability, a target protein Y-binding substance can also be used as a control.

“A target protein Y-binding substance” is a compound capable of interacting directly with target protein Y or a mutated protein thereof, and can be, for example, a protein, a nucleic acid, a carbohydrate, a lipid, or a small organic compound. Preferably, the target protein Y-binding substance can be selected from among thiabendazole, reserpine, imipenem, cephalexin, aclarubicin and derivatives thereof capable of binding to target protein Y (determined according to the kind of bioactive substance X) (described later), and salts thereof.

While the salts may be any salts, pharmaceutically acceptable salts are preferable; examples include salts with inorganic bases (e.g., alkali metals such as sodium and potassium; alkaline earth metals such as calcium and magnesium; aluminum, ammonium), salts with organic bases (e.g., trimethylamine, triethylamine, pyridine, picoline, ethanolamine, diethanolamine, triethanolamine, dicyclohexylamine, N,N-dibenzylethylenediamine), salts with inorganic acids (e.g., hydrochloric acid, hydrobromic acid, nitric acid, sulfuric acid, phosphoric acid), salt with organic acids (e.g., formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid), salts with basic amino acids (e.g., arginine, lysine, ornithine) or salts with acidic amino acids (e.g., aspartic acid, glutamic acid) and the like.

Furthermore, the binding ability may also be measured on the basis of the binding ability of target protein Y to each isoform (e.g., splicing variant) or the isoform-isoform binding ability ratio, rather than on the basis of the total binding ability of target protein Y.

The binding ability can also be measured in silico. For example, a measurement of the binding ability can be performed on the basis of SBDD (structure-based drug design: SBDD) or CADD (computer-aided drug design). Examples of such screening include virtual screening, de novo design, pharmacophore analysis, QSAR (quantitative structure activity relationship) and the like. If information on the steric structure of the protein or the target site of the protein is required during such screening, the information on the steric structure is used, provided that the steric structure is known by a structural analytical technique such as NMR, X-ray crystallographic analysis, or synchrotron radiation analysis. If the steric structure is unknown, information obtained by a structural prediction method such as the homology method or the threading method is used. In virtual screening, a program known per se can be used; examples of the program include DOCK (Kuntz, I. D. et al., Science, 1992, 257, 1078), Gold (Jones, G. et al., J. Mol. Biol., 1995, 245, 43), FlexX (Rarey, M. et al., J. Mol. Biol., 1996, 261, 470), AutoDock (Morris, G. M. et al., J. Comput. Chem., 1998, 19, 1639), ICM (Abagyan, R. A. et al., J. Comput. Chem., 1994, 15, 488) and the like.

In step (c) of methodology III, a test substance capable of binding to target protein Y is selected. The test substance capable of binding to the protein is capable of promoting or suppressing a function of target protein Y. The test substance thus selected can be useful for the regulation of a disease or condition associated with bioactive substance X.

In still another mode of embodiment, screening method Ia comprises the following steps (a), (b) and (c):

(a) a step for bringing the test substance and a target protein Y-binding substance into contact with target protein Y;
(b) a step for measuring the ability of the target protein Y-binding substance to bind to the protein in the presence of the test substance, and comparing this binding ability with the ability of the target protein Y-binding substance to bind to the protein in the absence of the test substance;
(c) a step for selecting a test substance that alters the ability of the target protein Y-binding substance to bind to the protein on the basis of the result of the comparison in step (b) above.

The methodology comprising the above-described steps (a) to (c) is referred to as “methodology IV” as required.

In step (a) of methodology IV, both a test substance and a target protein Y-binding substance are brought into contact with target protein Y. Contact of the test substance and the target protein Y-binding substance with the protein can be performed by mixing the test substance, the target protein Y-binding substance, and the protein in solution. The order of bringing the test substance and the target protein Y-binding substance into contact with the protein is not subject to limitation; one of them may be brought into contact with the protein at a time lag or at the same time.

Target protein Y can be prepared by a method known per se. For example, preparation of the protein can be performed by a method described in methodology III above.

The target protein Y-binding substance may be a labeled supply or a non-labeled supply, or a mixture of a labeled supply and a non-labeled supply mixed in a specified ratio. The labeling substance is the same as described above.

In step (b) of methodology IV, first, the ability of the target protein Y-binding substance to bind to the protein is measured in the presence of the test substance. “A binding ability” measured may be any one that enables an evaluation of the binding of the protein and the test substance; examples include binding amount, binding strength (including parameters such as affinity constant, binding rate constant, and dissociation rate constant), and binding mode (including dose-dependent binding).

A measurement of the binding ability can be performed using, for example, a labeled target protein Y-binding substance. The target protein Y-binding substance bound to the protein and the unbound target protein Y-binding substance may be separated before measuring the binding ability. More specifically, a measurement of the binding ability can be performed in the same manner as methodology III.

The binding ability may also be measured on the basis of the binding ability of target protein Y to each isoform (e.g., splicing variant) or the isoform-isoform binding ability ratio, rather than on the basis of the total amount of target protein Y bound.

Next, the binding ability of the target protein Y-binding substance to the protein in the presence of the test substance is compared with the binding ability of the target protein Y-binding substance to the protein in the absence of the test substance. This comparison of the binding ability is preferably performed on the basis of a significant difference. Although the binding ability of the target protein Y-binding substance to the protein in the absence of the test substance may be measured prior to, or simultaneously with, the measurement of the binding ability of the target protein Y-binding substance to the protein in the presence of the test substance, it is preferable, from the viewpoint of experimental accuracy and reproducibility, that the binding ability be measured simultaneously.

In step (c) of methodology IV, a test substance that alters the ability of the target protein Y-binding substance to bind to the protein is selected. The change in the binding ability can be, for example, a reduction or increase of binding ability, with preference given to a reduction of binding ability. The test substance thus selected can be useful for the regulation of an action associated with bioactive substance X.

Screening method Ia can further comprise (d) (i) a step for confirming that the test substance is capable of regulating, for example, promoting or suppressing, an action associated with bioactive substance X (confirmation step), or (ii) a step for identifying the kind of action exhibited by the test substance (identification step). The confirmation step or identification step can be performed by, for example, administering the test substance to a normal animal or an animal with “a disease or condition associated with bioactive substance X” or model animal. Alternatively, these steps can be performed by bringing the test substance into contact with the cells and evaluating the change in the phenotype of the cells after the contact. According to this identification step, the kind of “action associated with bioactive substance X” exhibited by the selected test substance can be determined, and whether or not the selected test substance can be used as either a drug or an investigational reagent, or both, and the kind of drug or investigational reagent to which the test substance is applicable can be confirmed.

Screening method Ia can also be performed by administration of the test substance to an animal. In this case, for example, not only the expression level of target gene Y, but also the expression level of target protein Y (e.g., abundance or intracellular amount of target protein Y in a specified tissue or cell of the animal to which the test substance has been administrated) can be measured. Examples of the animal include mammals such as mice, rats, hamsters, guinea pigs, rabbits, dogs, and monkeys, and birds such as chickens. When a screening method of the present invention is performed using an animal, for example, a test substance that regulates the expression level of target gene Y can be selected.

When screening method Ia is performed by administration of the test substance to the animal, intracellular localization of target protein Y in the animal may be measured. The measurement of the intracellular localization can be performed by the same method as described above.

Furthermore, provided that target gene Y is a gene for a soluble (secretory) factor, screening method Ia can be performed on the basis of a change in the blood concentration of the factor in the animal. Administration of the test substance to the animal, blood drawing from the animal, and the measurement of the blood concentration of the factor can be performed by a method known per se.

Screening method Ia enables screening of a substance capable of regulating an action associated with bioactive substance X. Hence, screening method Ia is useful for the development of a prophylactic or therapeutic agent for a disease or condition associated with bioactive substance X, an investigational reagent for the disease or the condition, and the like.

2.1.2. Screening Method for Substances Capable of Regulating an Action Associated with Bioactive Substance X, Which Comprises Selecting a Test Substance Incapable of Regulating the Expression or Function of Target Protein Y (Screening Method Ib)

The present invention provides a screening method for test substances capable of regulating an action associated with bioactive substance X (particularly an action associated with a known target molecule and/or a pharmacological action actually exhibited by bioactive substance X), which comprises determining whether or not a test substance is capable of regulating the expression or function of target protein Y, and selecting a test substance incapable of regulating the expression or function of target protein Y (e.g., a substance having a pharmacological action actually exhibited by bioactive substance X, which can be used for the same medicinal purpose as bioactive substance X, and shows no or decreased adverse effect actually exhibited by bioactive substance X).

Screening method Ib can be performed in the same manner as methodologies I to IV except that a test substance that does not cause a change or does not have the binding ability or regulatory capacity in step (c) of the above-described methodologies I to IV is selected.

In screening method Ib, the test substance used can be one capable of regulating the expression or function of a known target molecule (see, e.g., Table 8), or one having an action associated with bioactive substance X (particularly, a pharmacological action actually exhibited by bioactive substance X). Hence, screening method Ib can be used in combination with a screening method for substances capable of regulating an action associated with a known target molecule, which comprises determining whether or not the test substance is capable of regulating the expression or function of the known target molecule. The screening method for substances capable of regulating an action associated with a known target molecule can be performed in the same manner as the above-described screening method Ia. Alternatively, screening method Ib can be used in combination with a screening method for substances capable of regulating an action associated with bioactive substance X (particularly, a pharmacological action actually exhibited by bioactive substance X), which method comprises determining whether or not a test substance is capable of regulating the action associated with bioactive substance X. This type of screening method can be performed in the same manner as in step (d) of screening method Ia described above and using an animal or a cell.

TABLE 8

| | Example of known | |
|---|---|---|
| | candidate target | |
| Compound | molecule | Pubmed |
| reserpine | chromaffin granule | J Biol Chem. 1993 |
| | amine transporter | Jan 5; 268(1): 29-34 |
| | vesicular amine | Proc Natl Acad Sci USA. 1992 |
| | transporter (VAT) | Oct 15; 89(20): 9730-3 |
| | synaptic vesicular amine | J Physiol. 1999 Jun 1; 517 |
| | transporter | (Pt 2): 495-505 |
| | amine transporter from | J Biol Chem. 1990 |
| | bovine chromaffin | Mar 5; 265(7): 3961-6 |
| | granules | |
| | chromaffin granule | Biochemistry. 1990 |
| | biogenic amine | Jan 23; 29(3): 603-8 |
| | transporter | |
| | synaptic vesicular amine | J Physiol. 1997 |
| | transporter | Jun 1; 501 (Pt 2): 363-74 |
| | actin | Eur J Pharmacol. 1991 |
| | | Aug 14; 207(4): 305-10 |
| imipenem | penicillin-binding | Am J Med. 1985 Jun |
| | proteins (PBPs)2 | 7; 78(6A): 33-40 |
| | penicillin-binding | Eur J Clin Microbiol Infect |
| | proteins (PBPs)2 | Dis. 1998 Nov; 17(11): 754-60 |
| | sodium dependent | J Antimicrob Chemother. 2001 |
| | dopamine transporter | May; 47 (5): 665-70 |
| cephalexin | organic anion | Pharmacol Exp Ther. 1999 |
| | transporter-1 | Aug; 290(2): 672-7 |

Screening method Ib enables the development of drugs having an ability to regulate an action associated with a known target molecule and/or a pharmacological action actually exhibited by bioactive substance X, and showing decreased adverse effects of bioactive substance X. Hence, screening method Ib is useful for the improvement of existing drugs capable of regulating an action associated with a known target molecule and the like.

2.2. Screening Method for Substances Capable of Regulating a Function Associated with Target Protein Y (Screening Method II)

The present invention provides a screening method for substances capable of regulating a function associated with target protein Y, which comprises comparing the an ability of a test substance to bind to target protein Y or an action associated therewith an ability of bioactive substance X to bind to target protein Y or an action associated therewith.

This screening method is referred to as “screening method II” as required.

In one embodiment, screening method II comprises the following steps (a), (b) and (c):

(a) a step for bringing the test substance into contact with target protein Y;
(b) a step for measuring the functional level of the protein in the presence of the test substance, and comparing this functional level with the functional level of the protein in the presence of bioactive substance X;
(c) a step for selecting a test substance that alters the functional level of the protein on the basis of the result of the comparison in step (b) above.

The methodology comprising the above-described steps (a) to (c) is the same as methodology I except that the reference control for step (b) is not “the functional level of target protein Y in the absence of the test substance” but “the functional level of target protein Y in the presence of bioactive substance X”.

In another embodiment, screening method II comprises the following steps (a), (b) and (c):

(a) a step for bringing the test substance and cells enabling a measurement of the expression of target protein Y or a gene that encodes the protein into contact with each other;
(b) a step for measuring the expression level in the cells in contact with the test substance, and comparing this expression level with the expression level in control cells in contact with bioactive substance X;
(c) a step for selecting a test substance that regulates the expression level on the basis of the result of the comparison in step (b) above.

The methodology comprising the above-described steps (a) to (c) is the same as methodology II except that the reference control for step (b) is not “the expression level in control cells not in contact with the test substance” but “the expression level in control cells in contact with bioactive substance X”.

In still another mode of embodiment, screening method II comprises the following steps (a), (b) and (c):

(a) a step for bringing the test substance into contact with target protein Y;
(b) a step for measuring the ability of the test substance to bind to the protein, and comparing this binding ability with the ability of bioactive substance X to bind to the protein;
(c) a step for selecting a test substance capable of binding to the protein on the basis of the result of step (b) above.

The methodology comprising the above-described steps (a) to (c) is the same as methodology III except that the reference control for step (b) is “the ability of bioactive substance X to bind to target protein Y”.

Screening method II enables, for example, screening of substances capable of regulating a function associated with target protein Y, probes for target protein Y, and the like. Hence, screening method II is useful for the screening of prophylactic or therapeutic agents for diseases or conditions associated with target gene Y, screening of investigational reagents for the diseases or conditions, and the like.

### 2.3. Products Obtained by Screening Methods

The present invention provides products obtained by the above-described screening methods, for example, screening methods I and II.

A product provided by a screening method of the present invention can be a substance obtained by a screening method of the present invention, or a bioactivity regulator comprising a substance obtained by the screening method (described later).

A product provided by a screening method of the present invention is useful for, for example, the prevention or treatment of a disease or condition associated with bioactive substance X, or a disease or condition associated with target gene Y, or as an investigational reagent for the disease or the condition, and the like.

### 3. Regulators

The present invention provides bioactivity regulators each comprising a substance that regulates the expression or function of a target gene for a bioactive substance. The regulators of the present invention can be roughly divided into two types from the viewpoint of the bioactivity regulated: regulators of actions associated with bioactive substance X, and regulators of functions associated with target protein Y. The individual regulators are hereinafter described in detail.

3.1. Regulators of Actions Associated with Bioactive Substance X (Regulator I)

The present invention provides a type of regulators of actions associated with bioactive substance X, each of which comprises a substance that regulates the expression or function of target gene Y.

The regulators of this type are generically referred to as “regulator I” as required.

The substance that regulates the expression or function of target gene Y can be, for example, a substance that suppresses the expression of target gene Y. The expression refers to a state in which target gene Y translation product is produced and is localized at the action site thereof in a functional condition. Hence, the substance that suppresses the expression may be one that acts in any stage of gene transcription, post-transcriptional regulation, translation, post-translational modification, localization and protein folding and the like.

Specifically, the substance that suppresses the expression of target gene Y is exemplified by transcription suppressor, RNA polymerase inhibitor, RNA-degrading enzyme, protein synthesis inhibitor, nuclear translocation inhibitor, protein-degrading enzyme, protein denaturant and the like; to minimize the adverse effects on other genes and proteins expressed in the cells, it is important that the substance that suppresses the expression of target gene Y be capable of specifically acting on the target molecule.

An example of the substance that suppresses the expression of target gene Y is an antisense nucleic acid to a transcription product of target gene Y, specifically mRNA or initial transcription product. “An antisense nucleic acid” refers to a nucleic acid that consists of a base sequence capable of hybridizing to the target mRNA (initial transcription product) under physiological conditions for cells that express target mRNA (initial transcription product), and capable of inhibiting the translation of the polypeptide encoded by the target mRNA (initial transcription product) in a hybridized state. The kind of antisense nucleic acid may be DNA or RNA, or a DNA/RNA chimera. Because a natural type antisense nucleic acid easily undergoes degradation of the phosphoric acid diester bond thereof by a nucleolytic enzyme present in the cells, an antisense nucleic acid of the present invention can also be synthesized using a modified nucleotide of the thiophosphate type (P═O in phosphate linkage replaced with P═S), 2′-O-methyl type and the like which are stable to degrading enzymes. Other important factors for the designing of antisense nucleic acid include increases in water-solubility and cellular membrane permeability and the like; these can also be cleared by choosing appropriate dosage forms such as those using liposome or microspheres.

The length of antisense nucleic acid is not subject to limitation, as long as the antisense nucleic acid is capable of specifically hybridizing to the transcription product of target gene Y; the antisense nucleic acid may be of a sequence complementary to a sequence of about 15 bases for the shortest, or the entire sequence of the mRNA (initial transcription product) for the longest. Considering the ease of synthesis, antigenicity and other issues, for example, oligonucleotides consisting of about 15 bases or more, preferably about 15 to about 30 bases, can be mentioned.

The target sequence for the antisense nucleic acid may be any sequence that inhibits the translation of target gene Y or a functional fragment thereof by being hybridized to the antisense nucleic acid, and may be the entire sequence or a partial sequence of mRNA, or the intron moiety of the initial transcription product; when an oligonucleotide is used as the antisense nucleic acid, it is desirable that the target sequence be located between the 5′ terminus of the mRNA of target gene Y and the C terminus of the coding region thereof.

Furthermore, the antisense nucleic acid may be not only capable of hybridizing to a transcription product of target gene Y to inhibit its translation, but also binding to target gene Y in the form of double-stranded DNA to form a triple-strand (triplex) and inhibit the transcription to mRNA.

Another example of the substance that suppresses the expression of target gene Y is a ribozyme capable of specifically cleaving a transcription product of target gene Y, specifically mRNA or initial transcription product in the coding region (including the intron portion in the case of initial transcription product). “A ribozyme” refers to an RNA possessing enzyme activity to cleave nucleic acids. Because it has recently been shown that an oligo-DNA having the base sequence of the enzyme activity site also possesses nucleic acid cleavage activity, this term is herein used to mean a concept including DNA, as long as sequence specific nucleic acid cleavage activity is possessed. The most versatile ribozyme is self-splicing RNA, found in infectious RNAs such as those of viroid and virusoid; this self-splicing RNA is known to occur in some types, including hammerhead type and hairpin type. When ribozyme is used in the form of an expression vector comprising a DNA that encodes the same, a hybrid ribozyme wherein a sequence modified from tRNA is further linked to promote localization to cytoplasm may be used [Nucleic Acids Res., 29(13): 2780-2788 (2001)].

A still another example of the substance that suppresses the expression of target gene Y is a decoy nucleic acid. A decoy nucleic acid refers to a nucleic acid molecule that mimics a region to which a transcription regulatory factor binds; the decoy nucleic acid, which is the substance that suppresses the expression of target gene Y, can be a nucleic acid molecule that mimics a region to which a transcription activation factor for target gene Y binds.

Examples of the decoy nucleic acid include oligonucleotides modified to make them unlikely to undergo degradation in a body, such as oligonucleotides having a thiophosphoric diester bond wherein an oxygen atom in the phosphoric diester binding site is replaced with a sulfur atom (S-oligo), and oligonucleotides wherein the phosphoric diester bond is replaced with an uncharged methyl phosphate group, and the like. Although the decoy nucleic acid may completely match with the region to which a transcription activation factor binds, the degree of matching may be such that the transcription activation factor for target gene Y can bind the decoy nucleic acid. The length of the decoy nucleic acid is not subject to limitation, as long as the transcription activation factor binds thereto. The decoy nucleic acid may comprise a repeat of the same region.

Still another example of the substance that suppresses the expression of target gene Y is a double-stranded oligo-RNA, i.e. siRNA, which is complementary to a partial sequence (including the intron portion in the case of an initial transcription product) in the coding region of a transcription product of target gene Y, specifically, the mRNA or initial transcription product. It has been known that so-called RNA interference (RNAi), which is a phenomenon that if short double stranded RNA is introduced into cells, mRNA complementary to the RNA is degraded, occurs in nematodes, insects, plants and the like; recently, it has been found that this phenomenon also occurs in animal cells [Nature, 411(6836): 494-498 (2001)], which is drawing attention as an alternative technique to ribozymes. The siRNA used may be internally synthesized as described below, and a commercially available one may be used.

An antisense oligonucleotide and ribozyme can be prepared by determining the target sequence for a transcription product of target gene Y, specifically the mRNA or initial transcription product on the basis of the cDNA sequence or genomic DNA sequence of target gene Y, and by synthesizing a sequence complementary thereto using a commercially available automated DNA/RNA synthesizer (Applied Biosystems Company, Beckman Instruments Company and the like). A decoy nucleic acid and siRNA can be prepared by synthesizing a sense strand and an antisense strand in an automated DNA/RNA synthesizer, respectively, denaturing the chains in an appropriate annealing buffer solution at about 90 to about 95° C. for about 1 minute, and then annealing the chains at about 30 to about 70° C. for about 1 to about 8 hours. A longer double-stranded polynucleotide can be prepared by synthesizing a complementary oligonucleotide chain in alternative overlaps, annealing them, and then ligating them with ligase.

Another example of the substance that suppresses the expression of target gene Y is an antibody against target protein Y. The antibody may be a polyclonal antibody or a monoclonal antibody, and can be prepared by a well-known immunological technique. The antibody may also be a fragment of an antibody (e.g., Fab, F(ab′)₂), or a recombinant antibody (e.g., single-chain antibody). Furthermore, the nucleic acid that encodes the antibody (one functionally linked to a nucleic acid having promoter activity) is also preferable as the substance that suppresses the expression of target gene Y.

The polyclonal antibody can be acquired by, for example, subcutaneously or intraperitoneally administering target protein Y or a fragment thereof (as required, may be prepared as a complex crosslinked to a carrier protein such as bovine serum albumin or KLH (keyhole limpet hemocyanin)) as the antigen, along with a commercially available adjuvant (e.g., Freund's complete or incomplete adjuvant) to an animal about 2 to 4 times at intervals of 2 to 3 weeks (the antibody titer of partially drawn serum has been determined by a known antigen-antibody reaction and its elevation has been confirmed in advance), collecting whole blood about 3 to about 10 days after final immunization, and purifying the antiserum. As the animal to receive the antigen, mammals such as rats, mice, rabbits, goat, guinea pigs, and hamsters can be mentioned.

The monoclonal antibody can be prepared by, for example, a cell fusion method (e.g., Takeshi Watanabe, Saibou Yugouhou No Genri To Monokuronaru Koutai No Sakusei, edited by Akira Taniuchi and Toshitada Takahashi, “Monokuronaru Koutai To Gan—Kiso To Rinsho—”, pages 2-14, Science Forum Shuppan, 1985). For example, the factor is administered subcutaneously or intraperitoneally along with a commercially available adjuvant to a mouse 2 to 4 times, and about 3 days after final administration, the spleen or lymph nodes are collected, and leukocytes are collected. These leukocytes and myeloma cells (e.g., NS-1, P3X63Ag8 and the like) are cell-fused to obtain a hybridoma that produces a monoclonal antibody against the factor. This cell fusion may be performed by the PEG method [J. Immunol. Methods, 81(2): 223-228 (1985)], or by the voltage pulse method [Hybridoma, 7(6): 627-633 (1988)]. A hybridoma that produces the desired monoclonal antibody can be selected by detecting an antibody that binds specifically to the antigen from the culture supernatant using a widely known EIA or RIA method and the like. Cultivation of the hybridoma that produces the monoclonal antibody can be performed in vitro, or in vivo such as in mouse or rat ascitic fluid, preferably in mouse ascitic fluid, and the antibody can be acquired from the culture supernatant of the hybridoma and the ascitic fluid of the animal, respectively.

However, in view of therapeutic efficacy and safety in humans, the antibody of the present invention may be a chimeric antibody or a humanized or human type antibody. The chimeric antibody can be prepared with reference to, for example, “Jikken Igaku (extra issue), Vol. 6, No. 10, 1988”, Japanese Patent Kokoku Publication No. HEI-3-73280 and the like. The humanized antibody can be prepared with reference to, for example, Japanese Patent Kohyo Publication No. HEI-4-506458, Japanese Patent Kokai Publication No. SHO-62-296890 and the like. The human antibody can be prepared with reference to, for example, “Nature Genetics, Vol. 15, p. 146-156, 1997”, “Nature Genetics, Vol. 7, p. 13-21, 1994”, Japanese Patent Kohyo Publication No. HEI-4-504365, International Patent Application Publication No. WO94/25585, “Nikkei Science, June issue, pp. 40 to 50, 1995”, “Nature, Vol. 368, pp. 856-859, 1994”, Japanese Patent Kohyo Publication No. HEI-6-500233 and the like.

The substance that regulates the expression or function of target gene Y can also be a substance that suppresses a function of target gene Y.

Although the substance that suppresses a function of target gene Y is not subject to limitation, as long as it is capable of interfering with an action of target gene Y, it is important that the substance be capable of specifically acting on the target molecule to minimize the adverse effect on other genes and proteins. Examples of the substance that specifically suppresses a function of target gene Y include a dominant negative mutant of target protein Y and a nucleic acid that encodes the mutant (one functionally linked to a nucleic acid having promoter activity).

A dominant negative mutant of target protein Y refers to a mutant having the activity thereof reduced as a result of mutagenesis to target protein Y. The dominant negative mutant can indirectly inhibit the activity of natural target protein Y by competing therewith. The dominant negative mutant can be prepared by introducing a mutation to a nucleic acid that encodes target gene Y. Examples of the mutation include amino acid mutations in a functional domain that result in a decrease in the function responsible for the domain (e.g., deletion, substitution, and addition of one or more amino acids). The mutation can be introduced by a method known per se using PCR or a commonly known kit.

Provided that the substance that suppresses the expression of target gene Y is a nucleic acid molecule, the regulator of the present invention can have an expression vector that encodes the nucleic acid molecule as the active ingredient thereof. In the expression vector, an oligonucleotide or polynucleotide that encodes the above-described nucleic acid molecule must be functionally linked to a promoter capable of exhibiting promoter activity in the cells of the recipient mammal. Any promoter capable of functioning in the recipient mammal can be used; examples include viral promoters such as the SV40-derived early promoter, cytomegalovirus LTR, Rous sarcoma virus LTR, MoMuLV-derived LTR, and adenovirus-derived early promoter, and mammalian structural protein gene promoters such as the β-actin gene promoter, PGK gene promoter, and transferrin gene promoter, and the like.

The expression vector preferably comprises a transcription termination signal, that is, a terminator region, downstream of the oligo(poly)nucleotide that encodes the nucleic acid molecule. The expression vector may further comprise a selection marker gene for selecting transformant cells (genes that confer resistance to drugs such as tetracycline, ampicillin, kanamycin, hygromycin, and phosphinothricin, gene that compensate for auxotrophic mutation, and the like).

Although the basic backbone vector used as the expression vector is not subject to limitation, vectors suitable for administration to mammals such as humans include viral vectors such as retrovirus, adenovirus, adeno-associated virus, herpesvirus, vaccinia virus, poxvirus, poliovirus, Sindbis virus, and Sendai virus. Adenovirus has advantageous features, including the very high efficiency of transfection and possibility of introduction to non-dividing cells. Because incorporation of the introduced gene to host chromosome is very rare, however, gene expression is transient, usually lasting for about 4 weeks. In view of the sustainability of therapeutic effect, it is also preferable to use adeno-associated virus, which offers relatively high gene transduction efficiency, which can be introduced to non-dividing cells, and which can be incorporated in chromosomes via a inverted terminal repeat sequence (ITR).

The substance that regulates the expression or function of target protein Y can also be thiabendazole, reserpine, imipenem, cephalexin, aclarubicin or a derivative thereof capable of binding to target protein Y (described later), or a salt thereof.

Regulator I, in addition to a substance that regulates the expression or function of target gene Y, can comprise any carrier, for example, a pharmaceutically acceptable carrier.

Examples of the pharmaceutically acceptable carrier include, but are not limited to, excipients such as sucrose, starch, mannitol, sorbitol, lactose, glucose, cellulose, talc, calcium phosphate, and calcium carbonate; binders such as cellulose, methylcellulose, hydroxypropylcellulose, polypropylpyrrolidone, gelatin, gum arabic, polyethylene glycol, sucrose, and starch; disintegrants such as starch, carboxymethylcellulose, hydroxypropylstarch, sodium-glycol-starch, sodium hydrogen carbonate, calcium phosphate, and calcium citrate; lubricants such as magnesium stearate, Aerosil, talc, and sodium lauryl sulfate; flavoring agents such as citric acid, menthol, glycyrrhizin ammonium salt, glycine, and orange powder; preservatives such as sodium benzoate, sodium hydrogen sulfite, methyl paraben, and propyl paraben; stabilizers such as citric acid, sodium citrate, and acetic acid; suspending agents such as methylcellulose, polyvinylpyrrolidone, and aluminum stearate; dispersing agents such as surfactants; diluents such as water, physiological saline, and orange juice; base waxes such as cacao fat, polyethylene glycol, and kerosene, and the like.

Preparations suitable for oral administration include liquids comprising an effective amount of substance dissolved in a diluent such as water, physiological saline, or orange juice, capsules, sachets or tablets comprising an effective amount of substance in the form of solid or granules, suspensions comprising an effective amount of substance suspended in an appropriate dispersant, emulsions comprising a solution of an effective amount of substance dispersed in an appropriate dispersant and the like.

Preparations suitable for parenteral administration (e.g., subcutaneous injection, intramuscular injection, topical injection, intraperitoneal injection, and the like) include aqueous and non-aqueous isotonic sterile injection liquids, which may comprise an antioxidant, a buffer solution, a bacteriostatic agent, an isotonizing agent and the like. Other examples are aqueous and non-aqueous sterile suspensions, which may comprise a suspending agent, a solubilizer, a thickening agent, a stabilizer, an antiseptic and the like. The preparation can be included in a container in a unit dose or multiple doses like an ampoule or vial. It is also possible to lyophilize the active ingredient and a pharmaceutically acceptable carrier and preserve them in a state that only requires dissolving or suspending in a suitable sterile vehicle immediately before use.

The dose of regulator I varies depending on the activity and kind of the active ingredient, severity of the disease, the animal species to be the administration subject, drug acceptability, body weight and age of the administration subject, and the like, it is generally about 0.001 to about 500 mg/kg a day for an adult based on the amount of the active ingredient.

Regulator I enables the regulation, for example, suppression or promotion, of an action associated with bioactive substance X. Hence, regulator I is useful for the prophylaxis and treatment of a disease or condition associated with bioactive substance X, and as an investigational reagent for the disease or the condition, and the like.

3.2. Regulator of a Function Associated with Target Protein Y (Regulator II)

The present invention provides a regulator of a function associated with target protein Y, which comprises bioactive substance X.

This regulator is referred to as “regulator II” as required.

Bioactive substance X can be thiabendazole, reserpine, imipenem, cephalexin, aclarubicin or a derivative thereof capable of binding to target protein Y (described later), or a salt thereof.

Regulator II can comprise, in addition to bioactive substance X, any carrier, for example, a pharmaceutically acceptable carrier. The dose of regulator II is the same as that of regulator I.

Regulator II enables the regulation, for example, suppression or promotion, of a function associated with target protein Y. Hence, regulator II is useful for the prophylaxis and treatment of a disease or condition associated with target gene Y, and as an investigational reagent for the disease, and the like.

### 4. Derivative Production Method and Product Obtained by the Method

### 4.1. Derivative Production Method

The present invention provides a method of producing a bioactive substance derivative, which comprises derivatizing a bioactive substance so as to be able to regulate the expression or function of the target gene.

Derivatization means that a compound obtained by replacing a particular atom or group in a lead compound with another atom or group, or a compound obtained by subjecting a lead compound to an addition reaction, is virtually or actually synthesized. For example, the lead compound can be bioactive substance X.

The derivatization of bioactive substance X can be performed so that the regulatory capability for the expression or function of target gene Y is retained, and as required, in view of other properties of the derivative obtained, such as water-solubility/lipid-solubility, stability, dynamics, bioavailability, toxicity and the like. The derivatization of bioactive substance X can be performed so that, for example, the regulatory capability for the expression or function of target gene Y can be increased. The derivatization of bioactive substance X can also be performed so that a function associated with target protein Y can be regulated.

The derivatization of bioactive substance X such that the regulatory capability for the expression or function of target gene Y is retained can be performed on the basis of, for example, SBDD (structure-based drug design) and CADD (computer-aided drug design). Examples of the design include virtual screening, de novo design, pharmacophore analysis, QSAR (quantitative structure activity relationship) and the like. If information on the steric structure of the protein itself or the target site of the protein is required during such designing, information on the steric structure is used provided that the steric structure is known by a structural analytical technique such as NMR, X-ray crystallographic analysis, or synchrotron radiation analysis. If the steric structure is unknown, information obtained by a structural predictive method such as the homology method or the threading method is used. In virtual screening, a program known per se is used; examples of the program include DOCK (Kuntz, I. D. et al., Science, 1992, 257, 1078), Gold (Jones, G. et al., J. Mol. Biol., 1995, 245, 43), FlexX (Rarey, M. et al., J. Mol. Biol., 1996, 261, 470), AtutoDock (Morris, G. M. et al., J. Comput. Chem., 1998, 19, 1639), ICM (Abagyan, R. A. et al., J. Comput. Chem., 1994, 15, 488) and the like.

The derivatization of bioactive substance X such that the regulatory capacity for the expression or function of target gene Y is retained can also be performed on the basis of, for example, biological verification (in vitro or in vivo method). In this case, for example, the above-described methodologies I to IV can be used. Furthermore, one of the above-described methods such as SBDD and CADD, and biological verification may be used in combination.

The particular atom in bioactive substance X (a lead compound), which is substituted for producing the derivative, may be any atom present in the lead compound, exemplified by a hydrogen atom, a halogen atom (e.g., fluorine atom, chlorine atom, bromine atom, iodine atom), an oxygen atom, a sulfur atom, a nitrogen atom, a carbon atom and the like.

The particular group in bioactive substance X, which is substituted for producing the derivative, may be any group present in bioactive substance X, and can, for example, be a group having a molecular weight of 1 to 500, preferably 1 to 300, more preferably 1 to 200, most preferably 1 to 100. Examples of the particular group include an optionally substituted C₁ to C₈ hydrocarbon group, an optionally substituted C₁ to C₈ acyl group, an optionally substituted aromatic or non-aromatic C₃ to C₁₄ hydrocarbon cyclic group, or an optionally substituted aromatic or non-aromatic C₃ to C₁₄ heterocyclic group, an amino group, an amino group mono- or di-substituted by an alkyl group having 1 to 4 carbon atoms or an acyl group having 2 to 8 carbon atoms, an amidino group, a carbamoyl group, a carbamoyl group mono- or di-substituted by an alkyl group having 1 to 4 carbon atoms, a sulfamoyl group, a sulfamoyl group mono- or di-substituted by an alkyl group having 1 to 4 carbon atoms, a carboxyl group, an alkoxycarbonyl group having 2 to 8 carbon atoms, a hydroxy group, an alkoxy group having 1 to 6 carbon atoms optionally substituted by 1 to 3 halogen atoms, an alkenyloxy group having 2 to 5 carbon atoms optionally substituted by 1 to 3 halogen atoms, a cycloalkyloxy group having 3 to 7 carbon atoms, an aralkyloxy group having 7 to 9 carbon atoms, an aryloxy group having 6 to 14 carbon atoms, a thiol group, an alkylthio group having 1 to 6 carbon atoms optionally substituted by 1 to 3 halogen atoms, an aralkylthio group having 7 to 9 carbon atoms, an arylthio group having 6 to 14 carbon atoms, a sulfo group, a cyano group, an azido group, a nitro group, a nitroso group and the like.

The optionally substituted C₁ to C₈ hydrocarbon group can, for example, be an optionally substituted C₁ to C₈ alkyl group, an optionally substituted C₂ to C₈ alkenyl group, or an optionally substituted C₂ to C₈ alkynyl group.

The C₁ to C₈ alkyl group in the optionally substituted C₁ to C₈ alkyl group may be linear or branched, preferably having 1 to 6 carbon atoms; examples include methyl, ethyl, propyl, isopropyl, butyl, isobutyl, sec-butyl, tert-butyl and the like.

The C₂ to C₈ alkenyl group in the optionally substituted C₂ to C₈ alkenyl group may be linear or branched, preferably having 2 to 6 carbon atoms; examples include ethenyl, 1-propenyl, 2-propenyl, 2-methyl-1-propenyl, 1-butenyl, 2-butenyl, 3-butenyl and the like.

The C₂ to C₈ alkynyl group in the optionally substituted C₂ to C₈ alkynyl group may be linear or branched, preferably having 2 to 6 carbon atoms; examples include ethynyl, 1-propynyl, 2-propynyl, 1-buthynyl, 2-buthynyl, 3-buthynyl and the like.

The C₁ to C₈ acyl group in the optionally substituted C₁ to C₈ acyl group may be linear or branched, preferably having 2 to 6 carbon atoms; examples include formyl, acetyl, propinoyl, butanoyl, 2-methylpropinoyl and the like.

The aromatic C₃ to C₁₄ hydrocarbon cyclic group in the optionally substituted aromatic C₃ to C₁₄ hydrocarbon cyclic group may be monocyclic, bicyclic or tricyclic, preferably having 3 to 12 carbon atoms; examples include phenyl and naphthyl.

The non-aromatic C₃ to C₁₄ hydrocarbon cyclic group in the optionally substituted non-aromatic C₃ to C₁₄ hydrocarbon cyclic group may be saturated or unsaturated monocyclic, bicyclic or tricyclic, preferably having 3 to 12 carbon atoms; examples include cycloalkyl groups (e.g., cyclopropyl, cyclobutyl, cyclopentyl, cyclohexyl, cycloheptyl, cyclooctyl), cycloalkenyl groups (e.g., 2-cyclopenten-1-yl, 3-cyclopenten-1-yl, 2-cyclohexen-1-yl, 3-cyclohexen-1-yl), cycloalkadienyl groups (e.g., 2,4-cyclopentadien-1-yl, 2,4-cyclohexadien-1-yl, 2,5-cyclohexadien-1-yl) and the like.

The aromatic C₃ to C₁₄ heterocyclic group in the optionally substituted aromatic C₃ to C₁₄ heterocyclic group is a monocyclic, bicyclic or tricyclic aromatic heterocyclic group containing 1 to 5 hetero atoms selected from among oxygen atoms, sulfur atoms and nitrogen atoms, in addition to carbon atoms, as the ring-forming atoms, preferably having 3 to 12 carbon atoms. Examples of the monocyclic aromatic C₃ to C₁₄ heterocyclic group include furyl, thienyl, pyrrolyl, oxazolyl, isoxazolyl, thiazolyl, isothiazolyl, imidazolyl, pyrazolyl, oxadiazolyl, furazanyl, thiadiazolyl, triazolyl, tetrazolyl, pyridyl, pyrimidinyl, pyridazinyl, pyrazinyl, triazinyl and the like. Examples of the bicyclic or tricyclic aromatic heterocyclic group include benzofuranyl, isobenzofuranyl, benzo[b]thienyl, indolyl, isoindolyl, 1H-indazolyl, benzimidazolyl, benzooxazolyl, benzothiazolyl, 1H-benzotriazolyl, quinolyl, isoquinolyl, cinnolyl, quinazolyl, quinoxalinyl, phthaladinyl, naphthylizinyl, purinyl, pteridinyl, carbazolyl, α-carbonylyl, β-carbonylyl, γ-carbonylyl, acrydinyl, phenoxazinyl, phenothiazinyl, phenadinyl, phenoxathiinyl, thianthrenyl, indolidinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl and the like.

The non-aromatic C₃ to C₁₄ heterocyclic group in the optionally substituted non-aromatic C₃ to C₁₄ heterocyclic group is a monocyclic, bicyclic or tricyclic saturated or unsaturated heterocyclic group containing 1 to 5 hetero atoms selected from among oxygen atoms, sulfur atoms and nitrogen atoms, in addition to carbon atoms, as the ring-forming atoms, preferably having 3 to 12 carbon atoms; examples include oxiranyl, azetidinyl, oxetanyl, thietanyl, pyrrolidinyl, tetrahydrofuryl, tetrahydropyranyl, morpholinyl, thiomorpholinyl, piperazinyl, pyrrolidinyl, piperidino, morpholino, thiomorpholino and the like.

The kind of the substituent in any group optionally substituted can be the same as the particular group in bioactive substance X (described above), which is substituted for producing the derivative.

The number of particular atoms or groups in bioactive substance X, which is substituted for producing the derivative is any one, as long as the derivative produced is capable of regulating the expression or function of the gene Y, for example, as long as it is capable of binding to target protein Y, and can be, for example, 1 to 10, preferably 1 to 5, more preferably 1 to 3, further more preferably 1 to 2, most preferably 1.

The kind of a particular atom or group used for substitution (i.e., an atom or group introduced to the substitution site) can be the same as the particular atom or group in bioactive substance X, which is substituted for producing the derivative.

The atom or group added to bioactive substance X for producing the derivative (i.e., an atom or group used in the addition reaction) is an atom permitting an addition reaction, for example, an atom such as the hydrogen atom or the halogen atom, or a group capable of acting as a nucleophile or electrophile, out of the particular atoms or groups in bioactive substance X (described above), which is substituted for producing the derivative.

The number of atoms or groups added to bioactive substance X for producing the derivative is any one, as long as the derivative produced is capable of regulating the expression or function of the gene Y, for example, as long as it is capable of binding to target protein Y, and can be, for example, less than 6, preferably less than 4, more preferably 2 or 1.

The production method of the present invention is useful for, for example, the development of prophylactic or therapeutic agents for diseases or conditions associated with bioactive substance X or diseases or conditions associated with target gene Y, or investigational reagents for the diseases or the conditions, and the like.

### 4.2. Products Obtained by the Derivative Production Method

The present invention provides a product obtained by the above-described method of producing a derivative.

The product provided by the above-described production method can be bioactive substance X derivative obtained by the production method of the present invention, and a bioactivity regulator comprising the derivative (described above).

A product provided by the above-described production method is useful for, for example, the prophylaxis or treatment of a disease or condition associated with bioactive substance X, or a disease or condition associated with target gene Y, or as investigational reagents for the disease or the condition, and the like.

### 5. Complex and a Method of Producing the Same

The present invention provides a complex comprising a bioactive substance and a target protein therefor.

The bioactive substance can be, for example, the above-described bioactive substance X. Specifically, bioactive substance X can be thiabendazole, reserpine, imipenem, cephalexin, aclarubicin or a derivative thereof capable of binding to target protein Y. The kind of bioactive substance X can be selected as appropriate according to the kind of target protein Y.

The target protein for the bioactive substance can be, for example, the above-described target protein Y. Specifically, target protein Y can be FLJ10368-, FLJ12389-, FLJ12435-, FLJ12502-, FLJ12514-, FLJ14583- or FLJ31146-derived protein. The kind of target protein Y used to form the complex can be selected as appropriate according to the kind of bioactive substance X.

In one embodiment, the complex of the present invention can be a complex according to a combination of thiabendazole, reserpine, imipenem, cephalexin, aclarubicin or a derivative thereof capable of binding to a target protein and a target protein therefor.

In another embodiment, the complex of the present invention can be a complex according to a combination of FLJ10368-, FLJ12389-, FLJ12435-, FLJ12502-, FLJ12514-, FLJ14583- or FLJ31146-derived protein and a bioactive substance capable of binding to the protein.

The complex of the present invention can be preferably a complex according to any combination of (a1) to (a5) above or (b1) to (b7) above.

The present invention also provides a method of producing a complex comprising a bioactive substance and a target protein therefor, which comprises bringing the bioactive substance and the target protein therefor into contact with each other. This contact can be performed by, for example, mixing the bioactive substance and the target protein in solution.

The complex of the present invention and the method of producing the complex can be useful in, for example, performing the screening methods of the present invention or the derivative production method of the present invention, or in cases where the complex is structurally analyzed to extensively investigate the mode of interaction between a bioactive substance and a target protein thereof, and the like.

### 6. Kit

The present invention provides a kit comprising a bioactive substance or a salt thereof.

In one embodiment, the kit of the present invention comprises the following (i) and (ii):

(i) a bioactive substance or a salt thereof;
(ii) a target protein for a bioactive substance, a nucleic acid that encodes the protein, an expression vector comprising the nucleic acid, cells enabling a measurement of the expression of a target gene for the bioactive substance, or an expression vector comprising the transcription regulatory region of a target gene for the bioactive substance and a reporter gene functionally linked to the region.

Provided that the kit of the present invention comprises a target protein for a bioactive substance, the protein is not in the form of a complex with the bioactive substance.

The bioactive substance, the target protein and target gene therefor, and the combination of bioactive substance and target protein therefor are the same as those described above (see, e.g., “5. Complex, and a method of producing the same”). The expression vector, the cells enabling a measurement of the expression of a target gene for a bioactive substance, the transcription regulatory region of the target gene for the bioactive substance, and the reporter gene functionally linked to the region, are the same as those described above (see, e.g., “2. Screening method, and product obtained by the method”).

The above-described kit of the present invention can be useful in, for example, performing the screening methods of the present invention, the derivative production method of the present invention, and the complex production method of the present invention and the like.

### 7. Determination Methods and Determination Kits for the Onset or Risk of Onset of Disease or Condition

The present invention provides determination methods and determination kits for the onset or risk of onset of a specified disease or condition. The determination methods and determination kits of the present invention can be roughly divided into determination methods and determination kits based on measurement of the expression level, and determination methods and determination kits based on identification of the polymorphism. Furthermore, they can be classified into determination methods and determination kits for the onset or risk of onset of a disease or condition associated with bioactive substance X, and determination methods and determination kits for the onset or risk of onset of a disease or condition associated with target gene Y, from the viewpoint of the disease or condition for which a determination of the onset or risk of onset is desired. The individual determination methods and determination kits are hereinafter described in detail. As required, “the expression of target protein Y or the gene that encodes the protein” is sometimes referred to as “expression of target protein Y” or “expression of target gene Y”, and “function of target protein Y or a gene that encodes the protein” is sometimes referred to as “function of target protein Y” or “function of target gene Y” as required.

### 7.1. Determination Methods and Determination Kits for the Onset or Risk of Onset of Disease or Condition on the Basis of Measurement of the Expression Level of Target Gene Y

7.1.1. Determination Method for the Onset or Risk of Onset of Disease or Condition Associated with Bioactive Substance X on the Basis of Measurement of the Expression Level of Target Gene Y (Determination Method I)

The present invention provides a determination method for the onset or risk of onset of a disease or condition associated with bioactive substance X, which comprises measuring the expression level of target gene Y.

This determination method is referred to as “determination method I” as required.

In one embodiment, determination method I comprises the following steps (a) and (b):

(a) a step for measuring the expression level of target gene Y in a biological sample collected from an animal;
(b) a step for evaluating the onset or likelihood of onset of a disease or condition associated with bioactive substance X on the basis of the expression level of target gene Y.

The methodology comprising the above-described steps (a) to (b) is referred to as “methodology V” as required.

In step (a) of methodology V, the expression level of target gene Y in a biological sample collected from an animal is measured. Although the animal is not particularly limited, a mammal or a bird is preferable, with greater preference given to a mammal. Examples of the mammal include laboratory animals such as mice, rats, hamsters, guinea pigs, and rabbits, domestic animals such as swine, bovine, goat, horses, and sheep, companion animals such as dogs and cats, and primates such as monkeys, orangutans, chimpanzees, and humans. Examples of the bird include chicken.

The biological sample may be any sample containing a tissue expressing target gene Y, or any sample containing secreted target protein Y. The sample containing a tissue expressing target gene Y differs according to the kind of target gene Y. The tissue expressing target gene Y can be examined using, for example, H-Inv DB. The sample containing secreted target protein Y differs according to the kind of target gene Y, and can, for example, be blood, plasma, serum, saliva, cerebrospinal fluid, tear, or urine.

In this step, a biological sample collected from an animal in advance is used; of course, this methodology V can further comprise a step for collecting a biological sample from an animal. Collection of a biological sample from an animal can be performed by a method known per se.

The expression level of target gene Y can be measured by a method known per se with a product, for example, a transcription product or translation product, of target gene Y, as the subject. For example, the expression level of a transcription product can be measured by preparing total RNA from the cells, and performing RT-PCR, Northern blotting and the like. The expression level of a translation product can also be measured by preparing an extract from the cells, and performing an immunological technique. Useful immunological techniques include radioimmunoassay (RIA) method, ELISA method (Methods in Enzymol. 70: 419-439 (1980)), fluorescent antibody technique, and the like.

In step (b) of methodology V, a determination is made whether or not the animal is suffering from a disease or condition associated with bioactive substance X on the basis of the expression level of target gene Y. Specifically, first, the measured expression level of target gene Y is compared with the expression level of target gene Y in an animal that has not contracted the disease or condition associated with bioactive substance X (e.g., a normal animal). This comparison of expression level is preferably performed on the basis of the presence or absence of a significant difference. The expression level of target gene Y in an animal that has not contracted the disease or condition associated with bioactive substance X can be determined by a method known per se.

Next, on the basis of the result of the comparison of the expression level of target gene Y, a judgment is made whether or not the animal is possibly suffering from a disease or condition associated with bioactive substance X, or is likely or unlikely to suffer from the same in the future. The combination of a disease or condition associated with bioactive substance X and target gene Y is the same as described above. It is known that in animals that have contracted a particular disease, a change in the expression of the gene associated with the disease is often observed. It is also known that prior to the onset of a particular disease, a change in the expression of the particular gene is often observed. Hence, by analyzing the expression level of target gene Y, it is possible to determine the onset or likelihood of onset of the disease or condition associated with bioactive substance X.

Determination method I enables a determination of the presence or absence of a disease or condition associated with bioactive substance X, or the likelihood of contracting the disease or condition. Hence, determination method I is useful for, for example, the easy and early detection of the disease or condition and the like.

7.1.2. Determination Kit for the Onset or Risk of Onset of Disease or Condition Associated with Bioactive Substance X on the Basis of Measurement of Expression Level of Target Gene Y (Determination Kit I)

The present invention provides a determination kit that enables the easy conduct of determination method I.

This determination kit is referred to as “determination kit I” as required.

In one embodiment, determination kit I comprises the following (i) and (ii):

(i) a means capable of measuring the expression level of target gene Y;
(ii) a medium recording the relationship between a disease or condition associated with bioactive substance X and the expression level of target gene Y.

The kit may further comprise a means capable of collecting a biological sample from an animal, or a transcription product of target gene Y or target protein Y and the like.

The means capable of measuring the expression level of target gene Y is not subject to limitation, as long as it allows a quantitation of the expression level of target gene Y; for example, such means are roughly divided into means capable of quantifying target protein Y, and means capable of quantifying a transcription product of target gene Y. The means may be labeled with a labeling substance. Provided that the means is not labeled with a labeling substance, the determination kit of the present invention may further comprise the labeling substance. The labeling substance is the same as described above.

Specifically, the means capable of quantifying target protein Y include an antibody against target protein Y (described above), bioactive substance X and the like. The antibody against target protein Y and bioactive substance X may be provided in a form immobilized on a chip such as a plate.

Examples of the means capable of quantifying a transcription product of target gene Y include a nucleic acid probe for a transcription product of target gene Y, a primer pair capable of amplifying a transcription product of target gene Y and the like. The nucleic acid probe and primer pair may be provided along with a reagent for transcription product extraction.

The nucleic acid probe for the transcription product of target gene Y is not subject to limitation, as long as it enables a measurement of the amount of the transcription product of target gene Y. Although the probe may be any of DNA and RNA, preference is given to DNA in view of stability and the like. The probe may be single-stranded or double-stranded. Although the probe size is not subject to limitation, as long as it enables detection of the transcription product of target gene Y, the size is preferably about 15 to 1000 bp, more preferably about 50 to 500 bp. The probe may be provided in a form immobilized on a chip like a microarray.

A primer pair enabling the amplification of target gene Y is selected so that a nucleotide fragment of detectable size is amplified. The nucleotide fragment of detectable size can have a length of, for example, about 100 bp or more, preferably about 200 bp or more, more preferably about 500 bp or more. Although the primer size is not subject to limitation, as long as target gene Y can be amplified, it can be preferably about 15 to 100 bp, more preferably about 18 to 50 bp, further 35 more preferably about 20 to 30 bp. Provided that the means capable of quantifying a transcription product of target gene Y is a primer pair capable of amplifying target gene Y, the determination kit can further comprise a reverse transcriptase.

The medium recording the relationship between a disease or condition associated with bioactive substance X and the expression level of target gene Y can be one recording the difference in the expression level of target gene Y between an animal suffering from a disease or condition associated with bioactive substance X and a non-suffering animal. The medium can be a document or a computer-readable recording medium, for example, a flexible disc, CD, DVD, hard disk and the like. The expression level of target gene Y in an animal suffering from a disease or condition associated with bioactive substance X can be increased or decreased compared to an animal not suffering from the disease or the condition.

Any means can be used to collect a biological sample from an animal, as long as it allows the obtainment of the biological sample from the animal; examples include blood drawing instruments such as injectors, biopsy instruments such as biopsy needles and biopsy forceps, surgical instruments such as surgical knives and scissors, and the like.

The transcription product of target gene Y or target protein Y can be used as, for example, a control.

Determination kit I enables a determination of the presence or absence of a disease or condition associated with bioactive substance X, or the likelihood of contracting the disease or condition. Hence, determination kit I is useful for, for example, the easy and early detection of the disease or condition and the like.

### 7.2. Determination Methods and Determination Kits for the Risk of Onset of Disease or Condition on the Basis of Identification of Polymorphism of Target Gene Y

7.2.1. Determination Method for the Risk of Onset of Disease or Condition Associated with Bioactive Substance X on the Basis of Identification of Polymorphism of Target Gene Y (Determination Method II)

The present invention provides a determination method for the risk of onset of a disease or condition associated with bioactive substance X, which comprises identifying the polymorphism of target gene Y.

This determination method is referred to as “determination method II” as required.

In one embodiment, determination method II comprises the following steps (a) and (b):

(a) a step for identifying the polymorphism of target gene Y in a biological sample collected from an animal;
(b) a step for evaluating the likelihood of the onset of a disease or condition associated with bioactive substance X on the basis of the type of polymorphism.

The methodology comprising the above-described steps (a) to (b) is referred to as “methodology VI” as required.

In step (a) of methodology VI, the type of polymorphism of target gene Y in a biological sample collected from an animal is identified. The animal is the same as described above.

Although the biological sample used may be one described with respect to methodology V above, this methodology VI enables the use of any tissue containing genomic DNA such as hair, nails, skin or mucosa as the biological sample. In view of the ease of procurement, burden on the human body and the like, the biological sample is preferably a sample of hair, nails, skin, mucosa, blood, plasma, serum, saliva and the like.

In this step, a biological sample previously collected from an animal is used, but of course this methodology VI can further comprise a step for collecting a biological sample from an animal. Collection of a biological sample from an animal can be performed by a method known per se.

A polymorphism of target gene Y means a mutation found at a frequency in the nucleotide sequence of the genomic DNA comprising target gene Y in a certain population, and can be one or more DNA substitutions, deletions, or additions (e.g., SNP, haplotype) in the genomic DNA comprising target gene Y, and a repeat, inversion, translocation and the like of the genomic DNA. Various types of polymorphism of target gene Y are registered with known databases, for example, H-Inv DB and the like. The type of polymorphism of target gene Y used in this determination method is a mutation in a nucleotide sequence whose frequency differs between animals suffering from a disease or condition associated with bioactive substance X and non-suffering animals out of all types of polymorphism in target gene Y, and can be, for example, one that alters the expression of target gene Y or alters a function associated with target protein Y (e.g., the ability of target protein Y to bind to bioactive substance X). Such types of polymorphism can be determined by a method known per se such as linkage analysis.

A determination of the type of polymorphism can be performed by a method known per se. For example, the RFLP (restriction fragment length polymorphism) method, the PCR-SSCP (single-stranded DNA conformation polymorphism) analysis method, the ASO (allele specific oligonucleotide) hybridization method, the direct sequencing method, the ARMS (amplification refracting mutation system) method, the denaturing gradient gel electrophoresis method, the RNase A cleavage method, the DOL (dye-labeled oligonucleotide ligation) method, the TaqMan PCR method, the invader method, the MALDI-TOF/MS method (matrix assisted laser desorption-time of flight/mass spectrometry) method, the TDI (template-directed dye-terminator incorporation) method and the like can be used.

In step (b) of methodology VI, a determination of the likelihood of contracting a disease or condition associated with bioactive substance X in an animal is made on the basis of the type of polymorphism. The combination of a disease or condition associated with bioactive substance X and target gene Y is the same as described above. It is known that animals susceptible to a particular disease often have a particular type of polymorphism in the gene associated with the disease. Hence, it is possible to determine the likelihood of the onset of a disease or condition associated with bioactive substance X by polymorphism analysis.

Determination method II enables a determination of the likelihood of contracting a disease or condition associated with bioactive substance X. Hence, determination method II is useful for the provision of an incentive for improving one's lifestyle for the purpose of preventing the disease or condition and the like.

7.2.2. Determination Kit for the Risk of Onset of Disease or Condition Associated with Bioactive Substance X on the Basis of Identification of Polymorphism of Target Gene Y (Determination Kit II)

The present invention also provides a determination kit that enables the easy conduct of determination method II.

This determination kit is referred to as “determination kit II” as required.

In one embodiment, determination kit II comprises the following (i) and (ii):

(i) a means capable of identifying the polymorphism of target gene Y;
(ii) a medium recording the relationship between a disease or condition associated with bioactive substance X and the polymorphism of target gene Y.

The kit may further comprise a means capable of collecting of a biological sample from an animal, or a nucleic acid that encodes target gene Y having a particular type of polymorphism, a nucleic acid that encodes target gene Y not having a particular type of polymorphism and the like.

The means capable of identifying the polymorphism of target gene Y is not subject to limitation, as long as it is capable of determining the polymorphism of target gene Y. The means may be labeled with a labeling substance. Provided that the means is not labeled with a labeling substance, this kit may further comprise the labeling substance. The labeling substance is the same as described above.

Specifically, the means capable of identifying the polymorphism of target gene Y can be a nucleic acid probe enabling a specific identification of target gene Y having a particular type of polymorphism, or a primer pair capable of specifically amplifying target gene Y having a particular type of polymorphism. The nucleic acid probe and primer pair can be ones for a genomic DNA comprising target gene Y or for a transcription product of target gene Y. The nucleic acid probe and primer pair may be provided along with a transcription product or a reagent for genomic DNA extraction.

The nucleic acid probe enabling a specific identification of target gene Y having a particular type of polymorphism is not subject to limitation, as long as target gene Y having a particular type of polymorphism can be selected. Although the probe may be any of DNA and RNA, preference is given to DNA in view of stability and the like. The probe may be any of single-stranded and double-stranded. The probe size is preferably as short as possible to enable selecting of target gene Y having a particular type of polymorphism, and can be, for example, a size of about 15 to 30 bp. The probe may be provided in a form immobilized on a chip like a microarray. The probe enables, for example, ASO (allele specific oligonucleotide) hybridization method.

The primer pair capable of specifically amplifying target gene Y having a particular type of polymorphism is selected so that a nucleotide fragment of measurable size is amplified. Such a primer pair is designed so that, for example, a polymorphism site is present at the 3′ terminus of either primer. The nucleotide fragment of measurable size can, for example, have a length of about 100 bp or more, preferably about 200 bp or more, more preferably about 500 bp or more. The primer size is not subject to limitation, as long as target gene Y can be amplified, and can be preferably about 15 to 100 bp, more preferably about 18 to 50 bp, further more preferably about 20 to 30 bp. Provided that the means capable of identifying the polymorphism of target gene Y is a primer pair for a transcription product of target gene Y, the determination kit can further comprise a reverse transcriptase.

As another means capable of identifying the polymorphism of target gene Y, a restriction enzyme that recognizes a site of a particular type of polymorphism can be mentioned. This means enables polymorphism analysis by RFLP.

The medium recording the relationship between a disease or condition associated with bioactive substance X and the polymorphism of target gene Y can be one recording the difference in the nucleotide sequence of the genomic DNA comprising target gene Y between an animal suffering from the disease or condition associated with bioactive substance X and a non-suffering animal. For example, the medium can be a document or a computer-readable recording medium such as a flexible disk, CD, DVD, and hard disk.

The means capable of collecting a biological sample from an animal is the same as described above.

A nucleic acid that encodes target gene Y having a particular type of polymorphism, and a nucleic acid that encodes target gene Y not having a particular type of polymorphism can, for example, be used as controls.

Determination kit II enables a determination of the likelihood of contracting a disease or condition associated with bioactive substance X. Hence, determination kit II is useful for the provision of an incentive for improving one's lifestyle for the purpose of preventing the disease or condition and the like.

7.2.3. Method of Determining the Risk of Onset of Disease or Condition Associated with Target Gene Y on the Basis of Identification of Polymorphism of Target Gene Y (Determination Method III)

The present invention provides a determination method for the risk of onset of a disease or condition associated with target gene Y, which comprises identifying the polymorphism of target gene Y.

This determination method is referred to as “determination method III” as required.

In one embodiment, determination method III comprises the following steps (a) and (b):

(a) a step for determining the type of the polymorphism of target protein Y in a biological sample collected from an animal;
(b) a step for evaluating the likelihood of the onset of a disease or condition associated with target gene Y on the basis of the type of polymorphism.

In determination method III, the type of polymorphism used to determine the risk of onset alters the ability of target protein Y to bind to bioactive substance X. The type of polymorphism can be determined by a method known per se such as binding assay.

The methodology comprising steps (a) and (b) above in determination method III is the same as methodology VI except for the type of polymorphism of target gene Y to be identified.

Determination method III enables a determination of the likelihood of contracting a disease or condition associated with target gene Y. Hence, determination method III is useful for the provision of an incentive for improving one's lifestyle for the purpose of preventing the disease or condition and the like.

7.2.4. Determination Kit for the Risk of Onset of Disease or Condition Associated with Target Gene Y on the Basis of Identification of Polymorphism of Target Gene Y (Determination Kit III)

The present invention also provides a determination kit that enables the easy conduct of determination method III.

This determination kit is referred to as “determination kit III” as required.

In one embodiment, determination kit III comprises the following (i) and (ii):

(i) a means capable of identifying the polymorphism of target gene Y;
(ii) a medium recording the relationship between a disease or condition associated with target gene Y and the polymorphism of target gene Y.

The kit may further comprise a means capable of collecting of a biological sample from an animal, or a nucleic acid that encodes target gene Y having a particular type of polymorphism, a nucleic acid that encodes target gene Y not having a particular type of polymorphism and the like.

In determination kit III, the type of polymorphism used to determine the risk of onset is one that alters the ability of target protein Y to bind to bioactive substance X. The type of polymorphism can be determined by a method known per se such as binding assay.

The constituents of determination kit III are the same as those of determination kit II except for the type of polymorphism of target gene Y to be identified.

Determination kit III enables a determination of the likelihood of contracting a disease or condition associated with target gene Y. Hence, determination kit III is useful for the provision of an incentive for improving one's lifestyle for the purpose of preventing the disease or condition and the like.

### 8. Determination Methods and Determination Kits for Susceptibility to Bioactive Substances

The present invention provides determination methods and determination kits for susceptibility to a bioactive substance. The determination methods and determination kits of the present invention can be roughly divided into determination methods and determination kits based on measurement of expression level, and determination methods and determination kits based on identification of polymorphism. Furthermore, they are classified into determination methods and determination kits for a disease or condition associated with bioactive substance X, and determination methods and determination kits for a disease or condition associated with target gene Y, from the viewpoint of a disease or condition for which a determination of susceptibility is desired. The individual determination methods and determination kits are hereinafter described in detail.

### 8.1. Determination Methods and Determination Kits for Susceptibility to Bioactive Substances on the Basis of Measurement of the Expression Level of Target Gene Y

8.1.1. Determination Method for Susceptibility to Bioactive Substance X in Disease or Condition Associated with Bioactive Substance X on the Basis of Measurement of the Expression Level of Target Gene Y (Determination Method IV)

The present invention provides a determination method for susceptibility to bioactive substance X in a disease or condition associated with bioactive substance X, which comprises measuring the expression level of target gene Y.

This determination method is referred to as “determination method IV” as required.

In one embodiment, determination method IV comprises the following steps (a) and (b):

(a) a step for measuring the expression level of target gene Y in a biological sample collected from an animal;
(b) a step for predicting the effect of bioactive substance X on the basis of the expression level of target gene Y.

The methodology comprising the above-described steps (a) to (b) is referred to as “methodology VII” as required.

Step (a) of methodology VII is the same as step (a) of methodology V.

In step (b) of methodology VII, the possible effect of bioactive substance X on animals is evaluated on the basis of the expression level of target gene Y. Specifically, first, the measured expression level of target gene Y is checked against data on the correlation of the expression level of target gene Y and susceptibility to bioactive substance X. The correlation between the expression level of target gene Y and susceptibility to bioactive substance X can be determined by a method known per se.

Next, from the result of the comparison, susceptibility to bioactive substance X is estimated. The combination of bioactive substance X and target gene Y are the same as described above. It is considered that in animals expressing a target gene for a bioactive substance at high levels, their susceptibility to the bioactive substance is high (or low), and that in animals expressing the same at low levels, their susceptibility is low (or high). Hence, it is possible to determine the susceptibility of an animal to bioactive substance X by analyzing the expression level of target gene Y. For example, provided that bioactive substance X is a drug, the likelihood or unlikelihood of obtainment of desired effect of the drug, or the probability of onset of adverse effect of a drug, can be determined.

Determination method IV enables a determination of susceptibility to bioactive substance X. Hence, determination method IV is useful for, for example, the evaluation of an action of bioactive substance X on a particular animal, and the like.

8.1.2. Determination Kit for Susceptibility to Bioactive Substance X in Disease or Condition Associated with Bioactive Substance X on the Basis of Measurement of the Expression Level of Target Gene Y (Determination Kit IV)

The present invention provides a determination kit that enables the easy conduct of determination method IV.

This determination kit is referred to as “determination kit IV” as required.

In one embodiment, determination kit IV comprises the following (i) and (ii):

(i) a means capable of measuring the expression level of target gene Y;
(ii) a medium recording the relationship between the effect of bioactive substance X and the expression level of target gene Y.

The kit may further comprise a means capable of collecting of a biological sample from an animal, or a transcription product of target gene Y or target protein Y and the like.

The constituents of determination kit IV are the same as those of determination kit I except medium (ii).

The medium recording the relationship between the effect of bioactive substance X and the expression level of target gene Y can be one incorporating data on the correlation of the expression level of target gene Y and susceptibility to bioactive substance X. The expression level of target gene Y in an animal highly susceptible to bioactive substance X can increase (or decrease) compared to a less susceptible animal.

Determination kit IV enables the easy determination of susceptibility to bioactive substance X. Hence, determination method IV is useful for, for example, the evaluation of an action of bioactive substance X on a particular animal and the like.

### 8.2. Determination Methods and Determination Kits for Susceptibility to Bioactive Substance X on the Basis of Identification of Polymorphism of Target Gene Y

8.2.1. Determination Method for Susceptibility to Bioactive Substance X in Disease or Condition Associated with Bioactive Substance X on the Basis of Identification of Polymorphism of Target Gene Y (Determination Method V)

The present invention provides a determination method for susceptibility to bioactive substance X in a disease or condition associated with bioactive substance X, which comprises identifying the polymorphism of target gene Y.

This determination method is referred to as “determination method V” as required.

In one embodiment, determination method V comprises the following steps (a) and (b):

(a) a step for identifying the polymorphism of target gene Y in a biological sample collected from an animal;
(b) a step for predicting the effect of bioactive substance X in a disease or condition associated with target gene Y on the basis of the presence or absence of is a particular type of polymorphism.

The methodology comprising the above-described steps (a) to (b) is referred to as “methodology VIII” as required.

Step (a) of methodology VIII is the same as step (a) of methodology VII.

In step (b) of methodology VIII, the effect of bioactive substance X in a disease or condition associated with bioactive substance X is evaluated on the basis of the type of polymorphism of target gene Y. Specifically, first, the identified type of polymorphism of target gene Y is checked against data on the correlation of the type of polymorphism of target gene Y and susceptibility to bioactive substance X in a disease or condition associated with bioactive substance X. This correlation can be determined by a method known per se.

Next, from the result of the comparison, susceptibility to bioactive substance X in a disease or condition associated with bioactive substance X is estimated. The combination of bioactive substance X and target gene Y are the same as described above. It is known that in animals that are highly susceptible to a bioactive substance, a particular type of polymorphism is often observed in a target gene for the bioactive substance. Hence, it is possible to determine the susceptibility of an animal to bioactive substance X by analyzing polymorphism. For example, provided that bioactive substance X is a drug, the likelihood or unlikelihood of obtainment of desired effect of the drug, or the probability of onset of adverse reaction of a drug, can be determined.

Determination method V enables the easy determination of susceptibility to bioactive substance X in a disease or condition associated with bioactive substance X. Hence, determination method V is useful for, for example, the evaluation of an action of bioactive substance X in a disease or condition associated with bioactive substance X and the like.

8.2.2. Determination Kit for Susceptibility to Bioactive Substance X in Disease or Condition Associated with Bioactive Substance X on the Basis of Identification of Polymorphism of Target Gene Y (Determination Kit V)

The present invention also provides a determination kit that enables the easy conduct of determination method V.

This determination kit is referred to as “determination kit V” as required.

In one embodiment, determination kit V comprises the following (i) and (ii):

(i) a means capable of identifying the polymorphism of target gene Y;
(ii) a medium recording the relationship between the effect of bioactive substance X and the polymorphism of gene Y.

The kit may further comprise a means capable of collecting a biological sample from an animal, or a nucleic acid that encodes target gene Y having a particular type of polymorphism, a nucleic acid that encodes target gene Y not having a particular type of polymorphism and the like.

The constituents of determination kit V are the same as those of determination kit II except medium (ii).

The medium recording the relationship between the effect of active substance X and the polymorphism of gene Y can be one incorporating data on the correlation of susceptibility to bioactive substance X in a disease or condition associated with bioactive substance X and the type of polymorphism of target gene Y. The type of polymorphism of target gene Y in animals that are highly susceptible to bioactive substance X in a disease or condition associated with bioactive substance X can be one that encodes a protein that is more (or less) bindable to bioactive substance X than in animals that are less susceptible.

Determination kit V enables a determination of susceptibility to bioactive substance X in a disease or condition associated with bioactive substance X. Hence, determination kit V is useful for, for example, the evaluation of an action of bioactive substance X in a disease or condition associated with bioactive substance X and the like.

8.2.3. Determination Method for Susceptibility to Bioactive Substance X in Disease or Condition Associated with Target Gene Y on the Basis of Identification of Polymorphism of Target Gene Y (Determination Method VI)

The present invention provides a determination method for susceptibility to bioactive substance X in a disease or condition associated with target gene Y, which comprises identifying the polymorphism of target gene Y.

This determination method is referred to as “determination method VI” as required.

In one embodiment, determination method VI comprises the following steps (a) and (b):

(a) a step for determining the type of polymorphism of target protein Y in a biological sample collected from an animal;
(b) a step for predicting the effect of bioactive substance X in a disease or condition associated with target gene Y on the basis of the presence or absence of a particular type of polymorphism.

In this determination method, the type of polymorphism used to determine the susceptibility is one that alters the ability of target protein Y to bind to bioactive substance X. The type of polymorphism can be determined by a method known per se such as binding assay. Animals having a target gene comprising the type of polymorphism that potentiates or reduces the binding ability to the bioactive substance are thought to be highly (or poorly) susceptible to the bioactive substance; animals having a target gene comprising a type of polymorphism that reduces the binding ability are considered to be less (or more) susceptible. Hence, the susceptibility of an animal to bioactive substance X can be determined by analyzing the type of polymorphism.

The methodology comprising steps (a) and (b) above in determination method VI is the same as methodology VIII except for the type of polymorphism of target gene Y to be identified.

Determination method VI enables the easy determination of susceptibility to bioactive substance X in a disease or condition associated with bioactive substance X. Hence, determination method VI is useful for, for example, the evaluation of an action of bioactive substance X in a disease or condition associated with bioactive substance X and the like.

8.2.4. Determination Kit for Susceptibility to Bioactive Substance X in Disease or Condition Associated with Target Gene Y on the Basis of Identification of Polymorphism of Target Gene Y (Determination Kit VI)

The present invention also provides a determination kit that enables the easy conduct of determination method VI.

This determination kit is referred to as “determination kit VI” as required.

In one embodiment, determination kit VI comprises the following (i) and (ii):

(i) a means capable of identifying the polymorphism of target gene Y;
(ii) a medium recording the relationship between a disease or condition associated with target gene Y and the polymorphism of target gene Y.

The kit may further comprise a means capable of collecting a biological sample from an animal, or a nucleic acid that encodes target gene Y having a particular type of polymorphism, a nucleic acid that encodes target gene Y not having a particular type of polymorphism and the like.

In determination kit VI, the type of polymorphism used to determine the risk of onset is one that alters the ability of target protein Y to bind to bioactive substance X. The type of polymorphism can be determined by a method known per se such as binding assay.

The constituents of determination kit VI are the same as those of determination kit V except for the type of polymorphism of target gene Y to be identified.

Determination kit VI enables a determination of susceptibility to bioactive substance X in a disease or condition associated with bioactive substance X. Hence, determination kit VI is useful for, for example, the evaluation of an action of bioactive substance X in a disease or condition associated with bioactive substance X and the like.

The disclosures in all publications mentioned herein, including patents and patent application specifications, are incorporated by reference herein to the extent that all of them have been given expressly.

The present invention is hereinafter described in more detail by means of the following examples, which, however, are not to be construed as limiting the technical scope of the present invention.

### EXAMPLES

### Reference Example 1

### Method for Protein Expression from Human Full-Length cDNA Clones

### 1. Preparation of Expression Plasmids

Genes of interest in human full-length cDNA clones were subjected to BP reaction with a PCR cloning vector Gateway pDONR201 using Gateway system available from Invitrogen according to the kit's protocol to give an entry vector. pEU3-NII (TOYOBO) compatible with a cell-free protein synthesis system (PROTEIOS; TOYOBO) using wheat germ extracts was used as a source vector, from which a double-tag destination vector used as the destination vector of the Gateway system was prepared by introducing Gateway cassette with Gateway recombinant sequence into the pEU3-NII vector so that the Gateway system could be utilized, and further modifying the resulting vector by PCR method so that peptides having histidine and FLAG tag sequences in the N-terminal region of an expressed protein would be expressed.

The prepared double-tag destination vector and entry vector were used to conduct BP reaction using the Gateway system (Invitrogen) according to the protocol. The resulting product was transformed into Escherichia coli competent cells DH5a to select clones into which the expression vector was introduced. Plasmids were prepared from the obtained clones using QIAfilter Midi kit (QIAGEN) following the kit's protocol. The obtained plasmids were subjected to phenol/chloroform treatment and the inactivation treatment of RNase according to the PROTEIOS (TOYOBO) protocol to give purified expression plasmids.

### 2. Acquisition of Purified Proteins

Recombinant proteins were synthesized by the cell-free protein synthesis system (PROTEIOS; TOYOBO) using wheat germ extracts. mRNAs were prepared according to the PROTEIOS protocol from the expression plasmids obtained by the method described in paragraph 1 above. A 20 μg aliquot of the obtained mRNA was used to synthesize proteins in 2 wells of a 96-well microtiter plate according to the PROTEIOS protocol. The synthesized proteins were subjected to high-speed centrifugation treatment to remove precipitations. The obtained soluble fractions were purified using ANTI-FLAG M2 Affinity Gel (SIGMA) having an anti-FLAG tag antibody immobilized thereon and according to the protocol to give purified proteins.

### Reference Example 2

### Method for Determining Binding Dissociation Constant in Human Protein-Pharmaceutical Drug Interaction Using Biacore

The surface of a CM5 sensor chip for S51 (commercially available from Biacore) was converted to NTA using 1 M EDC, 1.33 M NHS, and 16 mg/ml AB-NTA (pH 9.2) to give an NTA sensor chip for S51. The proteins expressed in the wheat germ system and purified with a FLAG tag were immobilized on this chip. The immobilization was performed by sequentially injecting 0.5 M NiCl₂, 0.4 M EDC, 0.1 M EDC, a ligand (protein) solution, and 1 M ethanolamine (pH 8.5) into the passage system of Biacore S51. A running buffer used for the immobilization was PBS (pH 7.4). The ligand-immobilized sensor chip was used to conduct assay described below. A running buffer used was prepared by adding DMSO to a final concentration of 5% to HBS (10 mM HEPES and 150 mM NaCl, (pH 7.6)), 0.005% P₂0, and 100 μM mineral ion cocktail (Ca(OAc)₂, Zn(OAc)₂.2H₂O, Cu(OAc)₂.H₂O, Co(OAc)₂. 4H₂O, Mn(OAc)₂.4H₂O, Mg(OAc)₂.4H₂O, and FeCl₃.6H₂O). Compounds to be measured were prepared by making ½ serial dilutions (9 points) from 62.5 μM to 0.244 μM solutions. Solvents used for the compound solutions were prepared in the same composition as that of the running buffer. A solution containing only the solvents and free of the compound was prepared for zero-concentration measurement. For the correction (solvent correction) of the effect of DMSO contained in the compound solutions and the running buffer, the same solutions as the running buffer containing 3.8 to 5.1% DMSO (8 points) were prepared to perform correction based on the measurement results of these solutions. The Compound Characterization Assay program of Biacore S51 was conducted to measure the interaction between the immobilized ligands (proteins) and the analytes (compounds; 62.5 μM to 0.244 μM), followed by analysis with a specific software.

### Example 1

### Analysis of Interaction Between FLJ10368-Derived Protein and Thiabendazole

A protein was expressed and purified from FLJ10368 according to the method of Reference Example 1, and the interaction between thiabendazole and the protein expressed and purified from FLJ10368 was analyzed according to the method of Reference Example 2. As a result, the binding amount increased depending on the dose of thiabendazole, and the binding saturation was observed at high doses of thiabendazole. Therefore, thiabendazole was confirmed to specifically interact with the FLJ10368-derived protein. A binding dissociation constant calculated using Biacore S51 specific software was Kd=8.605×10⁻⁷ M.

From the foregoing, the interaction between thiabendazole and the FLJ10368-derived protein was clarified. Thus, the FLJ10368-derived protein was found to be a target protein for thiabendazole. Therefore, a new drug can be screened by making screening candidate substances interact with the FLJ10368-derived protein. Specifically, a new drug can be screened by constructing a system which detects the interaction between the FLJ10368-derived protein and a candidate substance according to, for example, the method of Reference Example 2.

### Example 2

### Analysis of Interaction Between FLJ10368-Derived Protein and Reserpine

A protein was expressed and purified from FLJ10368 according to the method of Reference Example 1, and the interaction between reserpine and the protein expressed and purified from FLJ10368 was analyzed according to the method of Reference Example 2. As a result, the binding amount was increased dose-dependently on reserpine, and the binding was observed to be saturated at high doses of reserpine. Therefore, reserpine was confirmed to specifically interact with the FLJ10368-derived protein. A binding dissociation constant calculated using Biacore S51 specific software was Kd=2.958×10⁻⁵ M.

From the foregoing, the interaction between reserpine and the FLJ10368-derived protein was clarified. Hence, the FLJ10368-derived protein was found to be a target protein for reserpine. Therefore, a new drug can be screened by making the FLJ10368-derived protein interact with screening candidate substances. Specifically, a new drug can be screened by constructing a system which detects the interaction between the FLJ10368-derived protein and a candidate substance according to, for example, the method of Reference Example 2.

### Example 3

### Analysis of Interaction Between FLJ12389-Derived Protein and Thiabendazole

A protein was expressed and purified from FLJ12389 according to the method of Reference Example 1, and the interaction between thiabendazole and the protein expressed and purified from FLJ12389 was analyzed according to the method of Reference Example 2. As a result, the binding amount was increased dose-dependently on thiabendazole, and the binding was observed to be saturated at high doses of thiabendazole. Therefore, thiabendazole was confirmed to specifically interact with the FLJ12389-derived protein. A binding dissociation constant calculated using Biacore S51 specific software was Kd=9.347×10⁻⁶ M.

From the foregoing, the interaction between thiabendazole and the FLJ12389-derived protein was clarified. Thus, the FLJ12389-derived protein was found to be a target protein for thiabendazole. Therefore, a new drug can be screened by making screening candidate substances interact with the FLJ12389-derived protein. Specifically, a new drug can be screened by constructing a system which detects the interaction between the FLJ12389-derived protein and a candidate substance according to, for example, the method of Reference Example 2.

### Example 4

### Analysis of Interaction Between FLJ12435-Derived Protein and Imipenem

A protein was expressed and purified from FLJ12435 according to the method of Reference Example 1, and the interaction between imipenem and the protein expressed and purified from FLJ12435 was analyzed according to the method of Reference Example 2. As a result, the binding amount was increased dose-dependently on imipenem, and the binding was observed to be saturated at high doses of imipenem. Therefore, imipenem was confirmed to specifically interact with the FLJ12435-derived protein. A binding dissociation constant calculated using Biacore S51 specific software was Kd=1.924×10⁻⁵ M.

From the foregoing, the interaction between imipenem and the FLJ12435-derived protein was clarified. Thus, the FLJ12435-derived protein was found to be a target protein for imipenem. Therefore, a new drug can be screened by making screening candidate substances interact with the FLJ12435-derived protein. Specifically, a new drug can be screened by constructing a system which detects the interaction between the FLJ12435-derived protein and a candidate substance according to, for example, the method of Reference Example 2.

### Example 5

### Analysis of Interaction Between FLJ12502-Derived Protein and Cephalexin

A protein was expressed and purified from FLJ12502 according to the method of Reference Example 1, and the interaction between cephalexin and the protein expressed and purified from FLJ12502 was analyzed according to the method of Reference Example 2. As a result, the binding amount was increased dose-dependently on cephalexin, and the binding was observed to be saturated at high doses of cephalexin. Therefore, cephalexin was confirmed to specifically interact with the FLJ12502-derived protein. A binding dissociation constant calculated using Biacore S51 specific software was Kd=5.98×10⁻⁶ M.

From the foregoing, the interaction between cephalexin and the FLJ12502-derived protein was clarified. Thus, the FLJ12502-derived protein was found to be a target protein for cephalexin. Therefore, a new drug can be screened by making screening candidate substances interact with the FLJ12502-derived protein. Specifically, a new drug can be screened by constructing a system which detects the interaction between the FLJ12502-derived protein and a candidate substance according to, for example, the method of Reference Example 2.

### Example 6

### Analysis of Interaction Between FLJ12514-Derived Protein and Aclarubicin

A protein was expressed and purified from FLJ12514 according to the method of Reference Example 1, and the interaction between aclarubicin and the protein expressed and purified from FLJ12514 was analyzed according to the method of Reference Example 2. As a result, the binding amount was increased dose-dependently on aclarubicin, and the binding was observed to be saturated at high doses of aclarubicin. Therefore, aclarubicin was confirmed to specifically interact with the FLJ12514-derived protein. A binding dissociation constant calculated using Biacore S51 specific software was Kd=6.115×10⁻⁶ M.

From the foregoing, the interaction between aclarubicin and the FLJ12514-derived protein was clarified. Thus, the FLJ12514-derived protein was found to be a target protein for aclarubicin. Therefore, a new drug can be screened by making screening candidate substances interact with the FLJ12514-derived protein. Specifically, a new drug can be screened by constructing a system which detects the interaction between the FLJ12514-derived protein and a candidate substance according to, for example, the method of Reference Example 2.

### Example 7

### Analysis of Interaction Between FLJ12514-Derived Protein and Thiabendazole

A protein was expressed and purified from FLJ12514 according to the method of Reference Example 1, and the interaction between thiabendazole and the protein expressed and purified from FLJ12514 was analyzed according to the method of Reference Example 2. As a result, the binding amount was increased dose-dependently on thiabendazole, and the binding was observed to be saturated at high doses of thiabendazole. Therefore, thiabendazole was confirmed to specifically interact with the FLJ12514-derived protein. A binding dissociation constant calculated using Biacore S51 specific software was Kd=2.318×10⁻⁶ M.

From the foregoing, the interaction between thiabendazole and the FLJ12514-derived protein was clarified. Thus, the FLJ12514-derived protein was found to be a target protein for thiabendazole. Therefore, a new drug can be screened by making screening candidate substances interact with the FLJ12514-derived protein. Specifically, a new drug can be screened by constructing a system which detects the interaction between the FLJ12514-derived protein and a candidate substance according to, for example, the method of Reference Example 2.

### Example 8

### Analysis of Interaction Between FLJ14583-Derived Protein and Cephalexin

A protein was expressed and purified from FLJ14583 according to the method of Reference Example 1, and the interaction between cephalexin and the protein expressed and purified from FLJ14583 was analyzed according to the method of Reference Example 2. As a result, the binding amount was increased dose-dependently on cephalexin, and the binding was observed to be saturated at high doses of cephalexin. Therefore, cephalexin was confirmed to specifically interact with the FLJ14583-derived protein. A binding dissociation constant calculated using Biacore S51 specific software was Kd=8.364×10⁻⁶ M.

From the foregoing, the interaction between cephalexin and the FLJ14583-derived protein was clarified. Thus, the FLJ14583-derived protein was found to be a target protein for cephalexin. Therefore, a new drug can be screened by making screening candidate substances interact with the FLJ14583-derived protein. Specifically, a new drug can be screened by constructing a system which detects the interaction between the FLJ14583-derived protein and a candidate substance according to, for example, the method of Reference Example 2.

### Example 9

### Analysis of Interaction Between FLJ14583-Derived Protein and Imipenem

A protein was expressed and purified from FLJ14583 according to the method of Reference Example 1, and the interaction between imipenem and the protein expressed and purified from FLJ14583 was analyzed according to the method of Reference Example 2. As a result, the binding amount was increased dose-dependently on imipenem, and the binding was observed to be saturated at high doses of imipenem. Therefore, imipenem was confirmed to specifically interact with the FLJ14583-derived protein. A binding dissociation constant calculated using Biacore S51 specific software was Kd=3.019×10⁻⁵ M.

From the foregoing, the interaction between imipenem and the FLJ14583-derived protein was clarified. Thus, the FLJ14583-derived protein was found to be a target protein for imipenem. Therefore, a new drug can be screened by making screening candidate substances interact with the FLJ14583-derived protein. Specifically, a new drug can be screened by constructing a system which detects the interaction between the FLJ14583-derived protein and a candidate substance according to, for example, the method of Reference Example 2.

### Example 10

### Analysis of Interaction Between FLJ31146-Derived Protein and Cephalexin

A protein was expressed and purified from FLJ31146 according to the method of Reference Example 1, and the interaction between cephalexin and the protein expressed and purified from FLJ31146 was analyzed according to the method of Reference Example 2. As a result, the binding amount was increased dose-dependently on cephalexin, and the binding was observed to be saturated at high doses of cephalexin. Therefore, cephalexin was confirmed to specifically interact with the FLJ31146-derived protein. A binding dissociation constant calculated using Biacore S51 specific software was Kd=6.604×10⁻⁶ M.

From the foregoing, the interaction between cephalexin and the FLJ31146-derived protein was clarified. Thus, the FLJ31146-derived protein was found to be a target protein for cephalexin. Therefore, a new drug can be screened by making screening candidate substances interact with the FLJ31146-derived protein. Specifically, a new drug can be screened by constructing a system which detects the interaction between the FLJ31146-derived protein and a candidate substance according to, for example, the method of Reference Example 2.

The binding intensities (Kd values) for the specific interactions of drugs and proteins confirmed in this Example are shown in Table 9.

TABLE 9

| FLJ No. | drug to be bonded | Kd Value |
|---|---|---|
| FLJ10368 | thiabendazole | 8.605 × 10⁻⁷ M |
| | reserpine | 2.958 × 10⁻⁵ M |
| FLJ12389 | thiabendazole | 9.347 × 10⁻⁶ M |
| FLJ12435 | imipenem | 1.924 × 10⁻⁵ M |
| FLJ12502 | cephalexin | 5.98 × 10⁻⁶ M |
| FLJ12514 | aclarubicin | 6.115 × 10⁻⁶ M |
| | thiabendazole | 2.318 × 10⁻⁶ M |
| FLJ14583 | cephalexin | 8.364 × 10⁻⁶ M |
| | imipenem | 3.019 × 10⁻⁵ M |
| FLJ31146 | cephalexin | 6.604 × 10⁻⁶ M |

### INDUSTRIAL APPLICABILITY

The target proteins and target genes of the present invention are useful for the development of bioactive substances, for example, drug discovery and the like. The screening methods of the present invention and the derivative production methods of the present invention are useful for the development of prophylactic or therapeutic agents for various diseases or conditions, and investigational reagents for the diseases or the conditions and the like. The regulators and derivatives of the present invention are useful for the prevention and treatment of various diseases or conditions, and as investigational reagents for the diseases or the conditions and the like. The complexes and kits of the present invention are useful for the screening methods of the present invention, the derivative production methods of the present invention and the like. The determination methods and determination kits of the present invention are useful for the evaluation of the onset or likelihood of onset of various diseases or conditions, evaluation of susceptibility to bioactive substances, and the like in animals.

The target proteins and target genes of the present invention enable the development of bioactive substances, for example, drug discovery and the like. The screening methods of the present invention and the derivative production method of the present invention enable the development of prophylactic or therapeutic agents for various diseases or conditions, and investigational reagents for the diseases or the conditions, and the like. The regulators and derivatives of the present invention enable the prophylaxis and treatment of various diseases or conditions, and the development of investigational reagents for the diseases or the conditions, and the like. The complexes and kits of the present invention enable the implementation of the screening methods of the present invention, the derivative production methods of the present invention and the like. The determination methods and determination kits of the present invention enable the evaluation of the onset or likelihood of onset of various diseases or conditions in animals, and the evaluation of the susceptibility of animals to bioactive substances and the like.

This application is based on a patent application No. 2005-255471 filed in Japan (filing date: Sep. 2, 2005), the contents of which are incorporated in full herein by this reference.

1. A method for screening a substance capable of regulating an action associated with bioactive substance X, which comprises determining whether or not a test substance is capable of regulating the expression or function of target protein Y or a gene that encodes the protein, wherein the combination of bioactive substance X and target protein Y is any of the following (a1) to (a5):
(a1) a combination of thiabendazole and FLJ10368-, FLJ12389- or FLJ12514-derived protein;
(a2) a combination of reserpine and FLJ10368-derived protein;
(a3) a combination of imipenem and FLJ12435- or FLJ14583-derived protein;
(a4) a combination of cephalexin and FLJ12502-, FLJ14583- or FLJ31146-derived protein;
(a5) a combination of aclarubicin and FLJI2514-derived protein.

2. The method according to claim 1, which comprises the following steps (a) to (c):
(a) a step for bringing the test substance into contact with target protein Y;
(b) a step for measuring the functional level of the protein in the presence of the test substance, and comparing said functional level with the functional level of the protein in the absence of the test substance;
(c) a step for selecting a test substance that alters the functional level of the protein on the basis of the result of the comparison in (b) above.

3. The method according to claim 1, which comprises the following steps (a) to (c):
(a) a step for bringing the test substance and cells allowing a measurement of the expression of target protein Y or a gene that encodes the protein into contact with each other;
(b) a step for measuring the expression level of the gene in the cells in contact with the test substance, and comparing said expression level with the expression level of the gene in control cells not in contact with the test substance;
(c) a step for selecting a test substance that regulates the expression level of the gene on the basis of the result of the comparison in (b) above.

4. The method according to claim 1, which comprises the following steps (a) to (c):
(a) a step for bringing the test substance into contact with target protein Y;
(b) a step for measuring the ability of the test substance to bind to the protein;
(c) a step for selecting a test substance capable of binding to the protein on the basis of the result from (b) above.

5. The method according to claim 1, which comprises the following steps (a) to (c):
(a) a step for bringing the test substance and a target protein Y-binding substance into contact with target protein Y;
(b) a step for measuring the ability of the target protein Y-binding substance to bind to the protein in the presence of the test substance, and comparing said ability with the ability of the target protein Y-binding substance to bind to the protein in the absence of the test substance;
(c) a step for selecting a test substance that alters the ability of the target protein Y-binding substance to bind to the protein on the basis of the result of the comparison in (b) above.

6. A method for screening a substance capable of regulating a function associated with target protein Y, which comprises comparing the ability of a test compound to bind to target protein Y or the action associated with the test compound, with the ability of bioactive substance X to bind to target protein Y or the action associated with the bioactive substance, wherein the combination of target protein Y and bioactive substance X is any of the following (b1) to (b7):
(b1) a combination of FLJ10368-derived protein and thiabendazole, reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ12502-derived protein and cephalexin or a derivative thereof capable of binding to the protein;
(b5) a combination of FLJ12514-derived protein and aclarubicin, thiabendazole or a derivative thereof capable of binding to the protein;
(b6) a combination of FLJ14583-derived protein and cephalexin, imipenem or a derivative thereof capable of binding to the protein;
(b7) a combination of FLJ31146-derived protein and cephalexin or a derivative thereof capable of binding to the protein.

7. A substance obtained by the method according to claim 1.

8. An agent of regulating a bioactivity, which comprises a substance obtained by the method according to claim 1.

9. An agent of regulating an action associated with bioactive substance X, which comprises a substance that regulates the expression or function of target protein Y or a gene that encodes the protein, wherein the combination of bioactive substance X and target protein Y is any of the following (a1) to (a5):
(a1) a combination of thiabendazole and FLJ10368-, FLJ12389- or FLJ12514-derived protein;
(a2) a combination of reserpine and FLJ10368-derived protein;
(a3) a combination of imipenem and FLJ12435- or FLJ14583-derived protein;
(a4) a combination of cephalexin and FLJ12502-, FLJ14583- or FLJ31146-derived protein;
(a5) a combination of aclarubicin and FLJ12514-derived protein.

10. The agent according to claim 9, wherein the substance that regulates the expression or function of target protein Y or a gene that encodes the protein is a substance that suppresses the expression or function of the gene.

11. The agent according to claim 10, wherein the substance that suppresses the expression or function of target protein Y or a gene that encodes the protein is an antisense nucleic acid, ribozyme, decoy nucleic acid, siRNA, antibody or a dominant negative mutant, or an expression vector thereof.

12. The agent according to claim 9, which comprises target protein Y, or an expression vector comprising a nucleic acid that encodes the protein.

13. An agent of regulating a function associated with target protein Y, which comprises bioactive substance X, wherein the combination of target protein Y and bioactive substance X is any of the following (b1) to (b7):
(b1) a combination of FLJ10368-derived protein and thiabendazole, reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ12502-derived protein and cephalexin or a derivative thereof capable of binding to the protein;
(b5) a combination of FLJ12514-derived protein and aclarubicin, thiabendazole or a derivative thereof capable of binding to the protein;
(b6) a combination of FLJ14583-derived protein and cephalexin, imipenem or a derivative thereof capable of binding to the protein;
(b7) a combination of FLJ31146-derived protein and cephalexin or a derivative thereof capable of binding to the protein.

14. A method of producing a derivative of bioactive substance X, which comprises the derivatizing bioactive substance X so as to be able to regulate the expression or function of target protein Y or a gene that encodes the protein, wherein the combination of bioactive substance X and target protein Y is any of the following (a1) to (a5):
(a1) a combination of thiabendazole and FLJ10368-, FLJ12389- or FLJ12514-derived protein;
(a2) a combination of reserpine and FLJ10368-derived protein;
(a3) a combination of imipenem and FLJ12435- or FLJ14583-derived protein;
(a4) a combination of cephalexin and FLJ12502-, FLJ14583- or FLJ31146-derived protein;
(a5) a combination of aclarubicin and FLJ12514-derived protein.

15. A method of producing a derivative of a substance capable of regulating a function associated with target protein Y, which comprises derivatizing bioactive substance X so as to be able to regulate the ability of bioactive substance X to bind to target protein Y, wherein the combination of target protein Y and bioactive substance X is any of the following (b1) to (b7):
(b1) a combination of FLJ10368-derived protein and thiabendazole, reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ12502-derived protein and cephalexin or a derivative thereof capable of binding to the protein;
(b5) a combination of FLJ12514-derived protein and aclarubicin, thiabendazole or a derivative thereof capable of binding to the protein;
(b6) a combination of FLJ14583-derived protein and cephalexin, imipenem or a derivative thereof capable of binding to the protein;
(b7) a combination of FLJ31146-derived protein and cephalexin or a derivative thereof capable of binding to the protein.

16. A bioactive substance derivative obtained by the method according to claim 14.

17. An agent of regulating a bioactivity, which comprises a bioactive substance derivative obtained by the method according to claim 14.

18. A complex comprising bioactive substance X and target protein Y thereof, wherein the combination of bioactive substance X and target protein Y is any of the following (a1) to (a5) or (b1) to (b7):
(a1) a combination of thiabendazole and FLJ10368-, FLJ12389- or FLJ12514-derived protein;
(a2) a combination of reserpine and FLJ10368-derived protein;
(a3) a combination of imipenem and FLJ12435- or FLJ14583-derived protein;
(a4) a combination of cephalexin and FLJ12502-, FLJ14583- or FLJ31146-derived protein;
(a5) a combination of aclarubicin and FLJ12514-derived protein;
(b1) a combination of FLJ10368-derived protein and thiabendazole, reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ12502-derived protein and cephalexin or a derivative thereof capable of binding to the protein;
(b5) a combination of FLJ12514-derived protein and aclarubicin, thiabendazole or a derivative thereof capable of binding to the protein;
(b6) a combination of FLJ14583-derived protein and cephalexin, imipenem or a derivative thereof capable of binding to the protein;
(b7) a combination of FLJ31146-derived protein and cephalexin or a derivative thereof capable of binding to the protein.

19. A method of producing the complex according to claim 18, which comprises bringing the bioactive substance and the target protein therefor into contact with each other.

20. A kit comprising the following (i) and (ii):
(i) bioactive substance X or a salt thereof,
(ii) target protein Y, a nucleic acid that encodes the protein, an expression vector comprising the nucleic acid, cells that enable a measurement of the expression of target protein Y or a gene that encodes the protein, or an expression vector comprising the transcription regulatory region of a gene that encodes target protein Y and a reporter gene functionally linked thereto;
wherein the combination of bioactive substance X and target protein Y is any of the following (a1) to (a5) or (b1) to (b7):
(a1) a combination of thiabendazole and FLJ10368-, FLJ12389- or FLJ12514-derived protein:
(a2) a combination of reserpine and FLJ10368-derived protein,
(a3) a combination of imipenem and FLJ12435- or FLJ14583-derived protein:
(a4) a combination of cephalexin and FLJ12502-, FLJ14583- or FLJ31146-derived protein:
(a5) a combination of aclarubicin and FLJ12514-derived protein
(b1) a combination of FLJ10368-derived protein and thiabendazole, reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ12502-derived protein and cephalexin or a derivative thereof capable of binding to the protein:
(b5) a combination of FLJ12514-derived protein and aclarubicin, thiabendazole or a derivative thereof capable of binding to the protein;
(b6) a combination of FLJ14583-derived protein and cephalexin, imipenem or a derivative thereof capable of binding to the protein;
(b7) a combination of FLJ31146-derived protein and cephalexin or a derivative thereof capable of binding to the protein.

21. A method for determining the onset or risk of onset of a disease or condition associated with an action of bioactive substance X, or the onset or risk of onset of a disease or condition associated with a function of target protein Y, which comprises the following steps (a) and (b):
(a) a step for measuring the expression level and/or polymorphism of target protein Y or a gene that encodes the protein in a biological sample collected from an animal;
(b) a step for evaluating the onset or likelihood of onset of the disease or condition on the basis of the measured expression level and/or polymorphism;
wherein the combination of bioactive substance X and target protein Y is any of the following (a1) to (a5) or (b1) to (b7):
(a1) a combination of thiabendazole and FLJ10368-, FLJ12389- or FLJ12514-derived protein;
(a2) a combination of reserpine and FLJ10368-derived protein;
(a3) a combination of imipenem and FLJ12435- or FLJ14583-derived protein;
(a4) a combination of cephalexin and FLJ12502-, FLJ14583- or FLJ31146-derived protein;
(a5) a combination of aclarubicin and FLJ12514-derived protein;
(b1) a combination of FLJ10368-derived protein and thiabendazole reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ12502-derived protein and cephalexin or a derivative thereof capable of binding to the protein;
(b5) a combination of FLJ12514-derived protein and aclarubicin, thiabendazole or a derivative thereof capable of binding to the protein;
(b6) a combination of FLJ14583-derived protein and cephalexin, imipenem or a derivative thereof capable of binding to the protein;
(b7) a combination of FLJ31146-derived protein and cephalexin or a derivative thereof capable of binding to the protein.

22. A kit for determining the onset or risk of onset of a disease or condition associated with an action of bioactive substance X, or the onset or risk of onset of a disease or condition associated with a function of target protein Y, which comprises the following (i) and (ii):
(i) a means capable of measuring the expression level and/or polymorphism of target protein Y or a gene that encodes the protein;
(ii) a medium recording the relationship between the disease or condition and the expression level and/or polymorphism;
wherein the combination of bioactive substance X and target protein Y is any of the following (a1) to (a5) or (b1) to (b7):
(a1) a combination of thiabendazole and FLJ10368-, FLJ12389- or FLJ12514-derived protein;
(a2) a combination of reserpine and FLJ10368-derived protein;
(a3) a combination of imipenem and FLJ12435- or FLJ14583-derived protein;
(a4) a combination of cephalexin and FLJ12502-, FLJ14583- or FLJ31146-derived protein;
(a5) a combination of aclarubicin and FLJ12514-derived protein
(b1) a combination of FLJ10368-derived protein and thiabendazole, reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ12502-derived protein and cephalexin or a derivative thereof capable of binding to the protein;
(b5) a combination of FLJ12514-derived protein and aclarubicin, thiabendazole or a derivative thereof capable of binding to the protein;
(b6) a combination of FLJ14583-derived protein and cephalexin, imipenem or a derivative thereof capable of binding to the protein;
(b7) a combination of FLJ31146-derived protein and cephalexin or a derivative thereof capable of binding to the protein.

23. A method for determining susceptibility to bioactive substance X in a disease or condition associated with an action of bioactive substance X, or disease or condition associated with a function of target protein Y, which comprises the following steps (a) and (b):
(a) a step for measuring the expression level and/or polymorphism of target protein Y or a gene that encodes the protein in a biological sample collected from an animal;
(b) a step for predicting the effect of the bioactive substance on the basis of the measured expression level and/or polymorphism;
wherein the combination of bioactive substance X and target protein Y is any of the following (a1) to (a5) or (b1) to (b7):
(a1) a combination of thiabendazole and FLJ10368-, FLJ12389- or FLJ12514-derived protein;
(a2) a combination of reserpine and FLJ10368-derived protein;
(a3) a combination of imipenem and FLJ12435- or FLJ14583-derived protein;
(a4) a combination of cephalexin and FLJ12502-, FLJ14583- or FLJ31146-derived protein;
(a5) a combination of aclarubicin and FLJ12514-derived protein
(b1) a combination of FLJ10368-derived protein and thiabendazole reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ2502-derived protein and cephalexin or a derivative thereof capable of binding to the protein;
(b5) a combination of FLJ12514-derived protein and aclarubicin, thiabendazole or a derivative thereof capable of binding to the protein
(b6) a combination of FLJ14583-derived protein and cephalexin imipenem or a derivative thereof capable of binding to the protein
(b7) a combination of FLJ31146-derived protein and cephalexin or a derivative thereof capable of binding to the protein.

24. A kit for determining susceptibility to bioactive substance X in a disease or condition associated with an action of bioactive substance X, or susceptibility to bioactive substance X in a disease or condition associated with a function of target protein Y, which comprises the following (i) and (ii):
(i) a means capable of measuring the expression level and/or polymorphism of target protein Y or a gene that encodes target protein Y;
(ii) a medium recording the relationship between the effect of bioactive substance X and the expression level and/or polymorphism of the gene;
wherein the combination of bioactive substance X and target protein Y is any of the following (a1) to (a5) or (b1) to (b7):
(a1) a combination of thiabendazole and FLJ10368-, FLJ12389- or FLJ12514-derived protein;
(a2) a combination of reserpine and FLJ10368-derived protein;
(a3) a combination of imipenem and FLJ12435- or FLJ14583-derived protein;
(a4) a combination of cephalexin and FLJ12502-, FLJ14583- or FLJ31146-derived protein;
(a5) a combination of aclarubicin and FLJ12514-derived protein;
(b1) a combination of FLJ10368-derived protein and thiabendazole, reserpine or a derivative thereof capable of binding to the protein;
(b2) a combination of FLJ12389-derived protein and thiabendazole or a derivative thereof capable of binding to the protein;
(b3) a combination of FLJ12435-derived protein and imipenem or a derivative thereof capable of binding to the protein;
(b4) a combination of FLJ12502-derived protein and cephalexin or a derivative thereof capable of binding to the protein;
(b5) a combination of FLJ12514-derived protein and aclarubicin, thiabendazole or a derivative thereof capable of binding to the protein;
(b6) a combination of FLJ14583-derived protein and cephalexin, imipenem or a derivative thereof capable of binding to the protein;
(b7) a combination of FLJ31146-derived protein and cephalexin or a derivative thereof capable of binding to the protein.

25. A substance obtained by the method according to claim 6.

26. An agent of regulating a bioactivity, which comprises a substance obtained by the method according to claim 6.

27. A bioactive substance derivative obtained by the method according to claim 15.

28. An agent of regulating a bioactivity, which comprises a bioactive substance derivative obtained by the method according to claim 15.
